SEQUENCE LISTING (15 sequences)

SEQ ID NO: 1 - protein, 883 aa, Artificial Sequence
  Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - protein, 883 aa, Artificial Sequence
  Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKALNIAQNTAWKINKKVLAVANVITKWKHCPVEDIPAIEREELPMKPEDIDMNPEALTAWKRAAAAVYRKDKARKSRRLSLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHWSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWEAVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDASHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 3 - protein, 883 aa, Artificial Sequence
  Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKALNIAQNTAWKINKKVLAVANVITKWKHCPVEDIPAIEREELPMKPEDIDMNPEALTAWKRAAAAVYRKDKARKSRRLSLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDASHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 4 - protein, 883 aa, Artificial Sequence
  Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRLSLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWEAVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDASHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 5 - protein, 883 aa, Artificial Sequence
  Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKALNIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWEAVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDASHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 6 - protein, 883 aa, Artificial Sequence
  Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKALNIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 7 - protein, 883 aa, Artificial Sequence
  Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRLSLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 8 - protein, 883 aa, Artificial Sequence
  Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHWSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 9 - protein, 883 aa, Artificial Sequence
  Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWEAVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 10 - protein, 883 aa, Artificial Sequence
  Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDASHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 11 - protein, 884 aa, Artificial Sequence
  Synthetic polypeptide
MNIIENIEKNDFSEIELAAIPFNTLADHYGSALAKEQLALEHESYELGERRFLKMLERQAKAGEIADNAAAKPLLATLLPKLTTRIVEWLEEYASKKGRKPSAYAPLQLLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 12 - protein, 874 aa, Artificial Sequence
  Synthetic polypeptide
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 13 - protein, 883 aa, Artificial Sequence
  Synthetic polypeptide
MTNVINAPKNDFSDIANAIQPYNILADHYGAHLAATQLELEHEAHTEGEKRFLKAMERQIKAGEFGDNAVAKPLLSSLAPKFIEAWNTWFTEVESKRGKRPVAYNLVQKVAPEAAAFITLKVTLACLTKEEYTNLQSVATKIGRSIEDELRFGRIRDEEAKHFKNHVQEALNKRVGIVYKKAFMQAVEGKMLDAGQLQTKWTTWTPEESIHVGVRMLELLIGSTGLVELHRPFAGNVEKDGEYIQLTEQYVDLLSKRAGALAAIAPMYQPCVVPPKPWTSPVGGGYWAAGRKPLSLVRTGSKKGLERYNDVYMPEVYKAVNIAQNTPWKINKKVLAVVNEIVNWKHCPVDDVPALERGELPVKPEDIDTNEVALKAWKKAASAIYRKEKARVSRRMSMEFMLGQANKFAQFKAIWFPMNMDWRGRVYAVPMFNPQGNDMTKGLLTLAKGKPIGVDGYYWLKIHGANTAGVDKVDFAERIKFIEDNHENIMSVAADPIANTWWAEQDSPFCFLAFCFEYAGVQHHGMNYNCSLPLAFDGSCSGIQHFSAMLRDEIGGRAVNLLPSKEVQDIYRIVAERVNEILKQDVINGTDNEVETVTNKDTGEITEKLKLGTKELAGQWLAYGVTRKVTKRSVMTLAYGSKEYGFRDQVLEDTIQPAIDDGKGLMFTQPNQAAGYMAKLIWNAVTVTVVAAVEAMNWLKSAAKLLAAEVKDKKTKEVLRKRCAVHWVTPDGFPVWQEYRKPVQTRLNLMFLGQIRLQPTVNTNKDSGIDARKQESGIAPNFVHSMDGSHLRMTVVRSNEVYGVESFALIHDSFGTIPADAGNLFKAVRETMVNTYEENDVLADFYDQFADQLHESQLDKMPEMPAKGSLDLQEILKSDFAFA

SEQ ID NO: 14 - protein, 882 aa, Artificial Sequence
  Synthetic polypeptide
MNVINAPKNDFSDIANAIQPYNILADHYGAQLAATQLELEHEAHTEGEKRFLKAMERQIKAGEFGDNAVAKPLLSSLAPKFIEAWNTWFTEVEAKRGKRPVAYNLVQKVAPEAAAFITLKVTLACLTKEEFTNLQSVATKIGRSIEDELRFGRIRDEEAKHFKNHVQEALNKRVGIVYKKAFMQAVEGKMLDAGQLQTKWTTWTPEESIHVGVRMLELLIGSTGLVELHRPFAGNVEKDGEYIQLTEQYVDLLSKRAGALAAIAPMYQPCVVPPKPWTSPVGGGYWAAGRKPLSLVRTGSKKGLERYNDVYMPEVYKAVNIAQNTPWKINKKVLAVVNEIVNWKHCPVEDVPALERGELPVKPEDIDTNEAALKAWKKAASAIYRKEKARVSRRMSMEFMLGQANKFAQFKAIWFPMNMDWRGRVYAVPMFNPQGNDMTKGLLTLAKGKPIGVDGYYWLKIHGANTAGVDKVDFAERIKFIDDNHENIMSVAADPIANTWWAEQDSPFCFLAFCFEYAGVQHHGMNYNCSLPLAFDGSCSGIQHFSAMLRDEVGGRAVNLLPSKEVQDIYRIVAERVNEMLREAVINGTDNEVETVTNKDTGEITEKLKLGTKELAGQWLAYGVTRKVTKRSVMTLAYGSKEYGFRDQVLEDTIQPAIDDGKGLMFTQPNQAAGYMAKLIWESVTVTVVAAVEAMNWLKSAAKLLAAEVKDKKTKEVLRKRCAVHWVTPDGFPVWQEYKKPVQTRLNLMFLGQIRLQPTVNTNKDSGIDARKQESGIAPNFVHSMDGSHLRMTVVRSNEVYGVESFALIHDSFGTIPADAGNLFKAVRETMVNTYEENDVLADFYEQFADQLHESQLDKMPEMPAKGSLDLQEILKSDFAFA

SEQ ID NO: 15 - protein, 884 aa, Artificial Sequence
  Synthetic polypeptide
MNIIENIEKNDFSEIELAAIPFNTLADHYGSALAREQLALEHESYELGERRFLKMLERQAKAGEIADNAAAKPLLATLLPKLTTRIVEWLEEYATKKGRKPVAYAPLQSLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNIAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVAHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHENYGIESFALIHDSFGTIPADAGKLFKAVRETMVITYENNDVLADFYDQFADQLHETQLDKMPPLPKKGNLNLQDILKSDFAFA

(19) United States
(12) Patent Application Publication
(10) Pub. No.: US 2021/0180034 A1
(43) Pub. Date: Jun. 17, 2021

(54) T7 RNA POLYMERASE VARIANTS

(71) Applicant: GreenLight Biosciences, Inc., Medford, MA (US)

(72) Inventor: Rachit JAIN, Medford, MA (US)

(73) Assignee: GreenLight Biosciences, Inc., Medford, MA (US)

(21) Appl. No.: 17/046,665

(22) PCT Filed: Apr. 9, 2019
(86) PCT No.: PCT/US2019/026561

§ 371 Date: Oct. 9, 2020

Related U.S. Application Data
(60) Provisional application No. 62/655,747, filed on Apr. 10, 2018.

(51) Int. Cl. C12N 9/12; C12N 15/10

(52) U.S. Cl. CPC C12N 9/1247; C12Y 207/07006; C12N 15/1068

(57) ABSTRACT

Provided herein are T7 RNA polymerase variants (T7 RNAP variants) having increased RNA polymerase activity and/or thermal stability, and methods of use thereof.

### RELATED APPLICATIONS

This application claims the benefit under 35 U.S.C. § 119(e) of U.S. provisional application No. 62/655,747, filed Apr. 10, 2018, the content of which is incorporated by reference herein in its entirety.

### BACKGROUND

Transcription of deoxyribonucleic acid (DNA) to ribonucleic acid (RNA) during gene expression is a fundamental cellular process that occurs when RNA polymerase attaches to a DNA template to begin polymerization of RNA. Essential cellular processes such as transcription generally occur at 37° C., and many enzymes including RNA polymerase are typically inactive at temperatures above 37° C. Accordingly, RNA polymerase variants having increased thermal stability at high temperatures are needed for RNA production at elevated temperatures.

### SUMMARY

The present disclosure provides T7 RNA polymerase (RNAP) variants with enhanced thermostability. Use of these variants for RNA synthesis reactions results in an improvement in RNA yield and product profile (relative to wild-type T7 RNAP, even at temperatures above 37° C.). These variants were rationally designed through the identification of individual mutations predicted to improve protein stability and minimize impact on affinity. Variants engineered to include specific combinations of those mutations were then tested to identify T7 RNAP variants with improved properties, for example, improved RNA production at elevated temperatures.

Thus, some embodiments of the present disclosure provide a T7 RNAP variant comprising at least one amino acid substitution in the amino acid sequence identified by SEQ ID NO: 1, wherein at least one amino acid substitution is at a position selected from the group consisting of I320, I396, F546, S684 and G788.

In some embodiments, the present disclosure provides a T7 RNAP variant comprising at least two amino acid substitutions in the amino acid sequence identified by SEQ ID NO: 1, wherein at least two amino acid substitutions are at positions selected from the group consisting of I320, I396, F546, S684, and G788.

In some embodiments, the T7 RNAP variant comprises amino acid substitutions at positions: I320, I396, F546, S684, and G788 (M1) of the amino acid sequence identified by SEQ ID NO: 1. In some embodiments, the T7 RNAP variant comprises amino acid substitutions at positions: I320, I396, and G788 (M2) of the amino acid sequence identified by SEQ ID NO: 1. In some embodiments, the T7 RNAP variant comprises amino acid substitutions at position: I396, S684, and G788 (M3) of the amino acid sequence identified by SEQ ID NO: 1. In some embodiments, the T7 RNAP variant comprises amino acid substitutions at positions: I320, S684, and G788 (M4) of the amino acid sequence identified by SEQ ID NO: 1. The amino acid sequence of SEQ ID NO: 1 is the wild-type sequence of T7 RNAP.

The present disclosure provides, in some embodiments, compositions, kits, systems, and methods comprising a T7 RNAP variant described herein. In some embodiments, the present disclosure provides methods for producing ribonucleic acid (RNA). These methods, in some embodiments, comprise combining a T7 RNA polymerase variant as provided herein with nucleoside triphosphates and a deoxyribonucleic acid (DNA) template encoding an RNA of interest, and producing the RNA of interest.

### BRIEF DESCRIPTION OF THE DRAWINGS

FIG. 1 is a graph showing double stranded RNA (dsRNA) product titer (ng/μL) produced by T7 RNA polymerase variants after 2 hours incubation at reaction temperature in a 25 μL reaction (n=3).

FIG. 2 is a graph showing comparison of % RNA produced by T7 RNA polymerase variants based on temperature.

### DETAILED DESCRIPTION

Bacteriophage T7 RNA polymerase (T7 RNAP) is a DNA-dependent RNA polymerase belonging to the DNA-polymerase I family. Wild-type T7 RNAP comprises 883 amino acids, corresponding to SEQ ID NO: 1. Wild-type T7 RNAP has polymerase activity at 37° C., pH 7.5, and is inactive at higher temperatures. Thus, some aspects of this disclosure provide T7 RNA polymerase variants having increased thermal stability and improved RNA production at high temperatures as compared to wild-type T7 RNA polymerase. T7 RNA polymerase variants disclosed herein may be used in a variety of methods performed at a temperature of 37° C. or greater.

### T7 RNA Polymerase Variants

Some aspects of this disclosure provide bacteriophage T7 RNA polymerase (T7 RNAP) variants that differ from the amino acid sequence of a naturally occurring T7 RNAP identified by SEQ ID NO: 1.

Provided herein, in some embodiments, are T7 RNAP variants, comprising at least one amino acid substitution in the amino acid sequence of SEQ ID NO: 1, wherein at least one substitution is at a position(s) selected from the group consisting of I320, I396, F546, S684 and G788.

In some embodiments, at least one amino acid substitution is at position I320 in the amino acid sequence identified by SEQ ID NO: 1. In some embodiments, at least one amino acid substitution is at position I396 in the amino acid sequence identified by SEQ ID NO: 1. In some embodiments, at least one amino acid substitution is at position F546 in the amino acid sequence identified by SEQ ID NO: 1. In some embodiments, at least one amino acid substitution is at position S684 in the amino acid sequence identified by SEQ ID NO: 1. In some embodiments, at least one amino acid substitution is at position G788 in the amino acid sequence identified by SEQ ID NO: 1.

Provided herein, in some embodiments, are T7 RNAP variants comprising at least two, at least three, or at least four amino acid substitutions in the amino acid sequence of SEQ ID NO: 1, wherein at least two, at least three, at least four amino acid, or all five substitutions are at positions selected from the group consisting of I320, I396, F546, S684, and G788.

In some embodiments, the T7 RNAP variant comprises at least two amino acid substitutions in the amino acid sequence of SEQ ID NO: 1, wherein at least two amino acid substitutions are at positions selected from the group consisting of I320, I396, F546, S684, and G788.

In some embodiments, at least two amino acid substitutions are at positions I320 and I396 in the amino acid sequence identified by SEQ ID NO: 1. In some embodiments, at least two amino acid substitutions are at positions I320 and F546 in the amino acid sequence identified by SEQ ID NO: 1. In some embodiments, at least two amino acid substitutions are at positions I320 and S684 in the amino acid sequence identified by SEQ ID NO: 1. In some embodiments, at least two amino acid substitutions are at positions I320 and G788 in the amino acid sequence identified by SEQ ID NO: 1. In some embodiments, at least two amino acid substitutions are at positions I396 and F546 in the amino acid sequence identified by SEQ ID NO: 1. In some embodiments, at least two amino acid substitutions are at positions I396 and S684 in the amino acid sequence identified by SEQ ID NO: 1. In some embodiments, at least two amino acid substitutions are at positions I396 and G788 in the amino acid sequence identified by SEQ ID NO: 1. In some embodiments, at least two amino acid substitutions are at positions F546 and S684 in the amino acid sequence identified by SEQ ID NO: 1. In some embodiments, at least two amino acid substitutions are at positions F546 and G788 in the amino acid sequence identified by SEQ ID NO: 1. In some embodiments, at least two amino acid substitutions are at positions S684 and G788 in the amino acid sequence identified by SEQ ID NO: 1.

In some embodiments, the T7 RNAP variant comprises at least three amino acid substitutions in the amino acid sequence identified by SEQ ID NO: 1, wherein at least three amino acid substitutions are at positions selected from the group consisting of I320, I396, F546, S684, and G788.

In some embodiments, at least three amino acid substitutions are at positions I320, I396, and F546 in the amino acid sequence identified by SEQ ID NO: 1. In some embodiments, at least three amino acid substitutions are at positions I320, I396 and S684 in the amino acid sequence identified by SEQ ID NO: 1. In some embodiments, at least three amino acid substitutions are at positions I320, I396, and G788 in the amino acid sequence identified by SEQ ID NO: 1. In some embodiments, at least three amino acid substitutions are at positions I320, F546, and S684 in the amino acid sequence identified by SEQ ID NO: 1. In some embodiments, at least three amino acid substitutions are at positions I320, F546, and G788 in the amino acid sequence identified by SEQ ID NO: 1. In some embodiments, at least three amino acid substitutions are at positions I320, S684, and G788 in the amino acid sequence identified by SEQ ID NO: 1. In some embodiments, at least three amino acid substitutions are at positions I396, F546, and S684 in the amino acid sequence identified by SEQ ID NO: 1. In some embodiments, at least three amino acid substitutions are at positions I396, S684, and G788 in the amino acid sequence identified by SEQ ID NO: 1. In some embodiments, at least three amino acid substitutions are at positions I396, F546, and G788 in the amino acid sequence identified by SEQ ID NO: 1. In some embodiments, at least three amino acid substitutions are at positions F546, S684, and G788 in the amino acid sequence identified by SEQ ID NO: 1.

In some embodiments, the T7 RNAP variant comprises at least four amino acid substitutions in the amino acid sequence identified by SEQ ID NO: 1, wherein at least two amino acid substitutions are at positions selected from the group consisting of I320, I396, F546, S684, and G788.

In some embodiments, at least four amino acid substitutions are at positions I320, I396, F546, and S684 in the amino acid sequence identified by SEQ ID NO: 1. In some embodiments, at least four amino acid substitutions are at positions I320, I396, F546, and G788 in the amino acid sequence identified by SEQ ID NO: 1. In some embodiments, at least four amino acid substitutions are at positions I320, F546, S684, and G788 in the amino acid sequence identified by SEQ ID NO: 1. In some embodiments, at least four amino acid substitutions are at positions I320, I396, S684, and G788 in the amino acid sequence identified by SEQ ID NO: 1. In some embodiments, at least four amino acid substitutions are at positions 1396, F546, S684, and G788 in the amino acid sequence identified by SEQ ID NO: 1.

In some embodiments, the T7 RNAP variant comprises five amino acid substitutions in the amino acid sequence identified by SEQ ID NO: 1, wherein the five amino acid substitutions are at positions I320, I396, F546, S684, and G788.

Certain amino acids may be substituted with another amino acid at positions I320, I396, F546, S684, and G788 in the amino acid sequence identified by SEQ ID NO: 1. For example, amino acid substitutions include, but are not limited to, I320L, I320V, I396L, I396V, F546W, F546Y, S684A, S684V, G788A, and G788V of the amino acid sequence identified by SEQ ID NO: 1.

Accordingly, the T7 RNAP variant, in some embodiments, has at least one amino acid substitution at a position selected from the group consisting of I320, I396, F546, S684 and G788 in SEQ ID NO: 1, wherein the amino acid substitution at position I320 is I320L or I320V, wherein the amino acid substitution at position I396 is I396L or I396V, wherein the amino acid substitution at position F546 is F546W or F546Y, wherein the amino acid substitution at position S684 is S684A or S684V, and/or wherein the amino acid substitution at position G788 is G788A or G788V.

In some embodiments, the T7 RNAP variant has at least two amino acid substitutions at a position selected from the group consisting of I320, I396, F546, S684, and G788 in SEQ ID NO: 1, wherein the amino acid substitution at position I320 is I320L or I320V, wherein the amino acid substitution at position I396 is I396L or I396V, wherein the amino acid substitution at position F546 is F546W or F546Y, wherein the amino acid substitution at position S684 is S684A or S684V, and/or wherein the amino acid substitution at position G788 is G788A or G788V.

In some embodiments, the T7 RNAP variant has at least three amino acid substitutions at a position selected from the group consisting of I320, I396, F546, S684, and G788 in SEQ ID NO: 1, wherein the amino acid substitution at position I320 is I320L or I320V, wherein the amino acid substitution at position I396 is I396L or I396V, wherein the amino acid substitution at position F546 is F546W or F546Y, wherein the amino acid substitution at position S684 is S684A or S684V, and/or wherein the amino acid substitution at position G788 is G788A or G788V.

In some embodiments, the T7 RNAP variant has at least four amino acid substitutions at a position selected from the group consisting of I320, I396, F546, S684, and G788 in SEQ ID NO: 1, wherein the amino acid substitution at position I320 is I320L or I320V, wherein the amino acid substitution at position I396 is I396L or I396V, wherein the amino acid substitution at position F546 is F546W or F546Y, wherein the amino acid substitution at position S684 is S684A or S684V, and/or wherein the amino acid substitution at position G788 is G788A or G788V.

In some embodiments, the T7 RNAP variant has at least five amino acid substitutions at a position selected from the group consisting of I320, I396, F546, S684, and G788 in SEQ ID NO: 1, wherein the amino acid substitution at position I320 is I320L or I320V, wherein the amino acid substitution at position I396 is I396L or I396V, wherein the amino acid substitution at position F546 is F546W or F546Y, wherein the amino acid substitution at position S684 is S684A or S684V, and/or wherein the amino acid substitution at position G788 is G788A or G788V.

In some embodiments, the T7 RNAP variant comprises at least two amino acid substitutions in the amino acid sequence identified by SEQ ID NO: 1, wherein the at least two amino acid substitutions are selected from the group consisting of I320L, I320V, I396L, I396V, F546W, F546Y, S684A, S684V, G788A, and G788V. In some embodiments, the T7 RNAP variant comprises at least three amino acid substitutions in the amino acid sequence identified by SEQ ID NO: 1, wherein the at least three amino acid substitutions are selected from the group consisting of I320L, I320V, I396L, I396V, F546W, F546Y, S684A, S684V, G788A, and G788V. In some embodiments, the T7 RNAP variant comprises at least four amino acid substitutions in the amino acid sequence identified by SEQ ID NO: 1, wherein the at least four amino acid substitutions are selected from the group consisting of I320L, I320V, I396L, I396V, F546W, F546Y, S684A, S684V, G788A, and G788V. In some embodiments, the T7 RNAP variant comprises at least five amino acid substitutions in the amino acid sequence identified by SEQ ID NO: 1, wherein the at least five amino acid substitutions are selected from the group consisting of I320L, 1320V, I396L, I396V, F546W, F546Y, S684A, S684V, G788A, and G788V.

In some embodiments, the T7 RNAP variant comprises at least two amino acid substitutions in the amino acid sequence identified by SEQ ID NO: 1, wherein the at least two amino acid substitutions are selected from the group consisting of I320L, I396L, F546W, S684A, and G788A. In some embodiments, the T7 RNAP variant comprises at least three amino acid substitutions in the amino acid sequence identified by SEQ ID NO: 1, wherein the at least three amino acid substitutions are at positions selected from I320L, I396L, F546W, S684A, and G788A. In some embodiments, the T7 RNAP variant comprises at least four amino acid substitutions in the amino acid sequence identified by SEQ ID NO: 1, wherein the at least four amino acid substitutions are selected from the group consisting of I320L, I396L, F546W, S684A, and G788A.

In some embodiments, the T7 RNAP variant comprising I320L, I396L, and G788A amino acid substitutions comprises the amino acid sequence identified by SEQ ID NO: 3. In some embodiments, the T7 RNAP variant comprising I396L, S684A, and G788A amino acid substitutions comprises the amino acid sequence identified by SEQ ID NO: 4. In some embodiments, the T7 RNAP variant comprising I320L, S684A, and G788A amino acid substitutions comprises the amino acid sequence identified by SEQ ID NO: 5. In some embodiments, the T7 RNAP variant comprising I320L, I396L, F546W, S684A, and G788A amino acid substitutions comprises the amino acid sequence identified by SEQ ID NO: 2.

In some embodiments, the T7 RNAP variant comprises the amino acid sequence identified by SEQ ID NO: 3. In some embodiments, the T7 RNAP variant comprises the amino acid sequence identified by SEQ ID NO: 4. In some embodiments, the T7 RNAP variant comprises the amino acid sequence identified by SEQ ID NO: 5. In some embodiments, the T7 RNAP variant comprises the amino acid sequence identified by SEQ ID NO: 2.

The present disclosure encompasses T7 RNAP variants further comprising at least one substitution of an amino acid that is not at position I320, I396, F546, S684, or G788 of SEQ ID NO: 1. In some embodiments, the additional amino acid substitution(s) is not made at conserved amino acids, or at amino acids residing within a conserved motif, where such residues are essential for protein activity. In some embodiments, the additional an amino acid substitution may, however, be incorporated into a non-conserved region of a T7 RNAP variant such that the T7 RNAP variant retains its activity. In some embodiments, the T7 RNA variants of the present disclosure further comprise at least one amino acid substitution that is not described herein, provided the additional amino acid substitute does not inhibit polymerase activity. Thus, in some embodiments, a T7 RNA variant comprises an amino acid substitution at position I320 and at least one additional amino acid substitution. In some embodiments, a T7 RNA variant comprises an amino acid substitution at position I396 and at least one additional amino acid substitution. In some embodiments, a T7 RNA variant comprises an amino acid substitution at position F546 and at least one additional amino acid substitution. In some embodiments, a T7 RNA variant comprises an amino acid substitution at position S684 and at least one additional amino acid substitution. In some embodiments, a T7 RNA variant comprises an amino acid substitution at position G788 and at least one additional amino acid substitution.

The amino acid substitutions at positions I320, I396, F546, S684, and/or G788, in some embodiments, are incorporated into an amino acid sequence having at least 70%, at least 75%, at least 80%, at least 85%, at least 90%, at least 95%, at least 98%, or at least 99% identity to SEQ ID NO: 1. Accordingly, a T7 RNAP variant, in some embodiments, comprises two, three, four, or five amino acid substitutions at positions selected from I320, 1396, F546, S684, and G788 in an amino acid sequence having at least 70%, at least 75%, at least 80%, at least 85%, at least 90%, at least 95%, at least 98%, or at least 99% identity to SEQ ID NO: 1.

The term “identity” refers to a relationship between the sequences of two or more polypeptides, as determined by comparing (aligning) the sequences. Identity measures the percent of identical matches between the smaller of two or more sequences with gap alignments (if any) addressed by a particular mathematical model or computer program (e.g., “algorithms”). Identity of related molecules can be readily calculated by known methods. “Percent (%) identity” as it applies to amino acid sequences is defined as the percentage of amino acid residues in the candidate amino acid sequence that are identical with the residues in the amino acid sequence of a second sequence after aligning the sequences and introducing gaps, if necessary, to achieve the maximum percent identity. Identity depends on a calculation of percent identity but may differ in value due to gaps and penalties introduced in the calculation.

The comparison of sequences and determination of percent identity between two sequences can be accomplished using a mathematical algorithm. Techniques for determining identity are codified in publicly available computer programs. Exemplary computer software to determine homology between two sequences include, but are not limited to, GCG program package (Devereux, J. et al., Nucleic Acids Research, 12(1): 387, 1984), the BLAST suite (Altschul, S. F. et al., Nucleic Acids Res. 25: 3389, 1997), and FASTA (Altschul, S. F. et al., J. Molec. Biol. 215: 403, 1990). Other techniques include: the Smith-Waterman algorithm (Smith, T. F. et al., J. Mol. Biol. 147: 195, 1981; the Needleman-Wunsch algorithm (Needleman, S. B. et al., J. Mol. Biol. 48: 443, 1970; and the Fast Optimal Global Sequence Alignment Algorithm (FOGSAA) (Chakraborty, A. et al., Sci Rep. 3: 1746, 2013).

It should be understood that amino acid substitutions at positions I320, I396, F546, S684, and G788 may be transferred to other RNA polymerases with similar effects on the activity and thermal stability of the RNA polymerase. Examples of RNA polymerases include, but are not limited to, RNA polymerase from Bacteriophage T3 (NCBI Reference Sequence: NP_523301.1, SEQ ID NO: 11), RNA polymerase from Bacteriophage SP6 (UniProt: P06221, SEQ ID NO: 12), RNA Polymerase from Erwinia phage FE44 (NCBI Reference Sequence: YP_008766719.1, SEQ ID NO: 13), RNA polymerase from Kluyvera bacteriophage Kvp1 (GenBank: ACJ14548.1, SEQ ID NO: 14), and RNA polymerase from Yersinia bacteriophage phiYeO3-12 (UniProt: Q9T145, SEQ ID NO: 15).

In some aspects of this disclosure, nucleic acids encoding a T7 RNAP variant are provided herein. In some embodiments, the nucleic acid encodes a T7 RNAP variant of SEQ ID NO: 2. In some embodiments, the nucleic acid encodes a T7 RNAP variant of SEQ ID NO: 3. In some embodiments, the nucleic acid encodes a T7 RNAP variant of SEQ ID NO: 4. In some embodiments, the nucleic acid encodes a T7 RNAP variant of SEQ ID NO: 5.

### RNA Polymerase Activity and Thermal Stability

Some aspects of this disclosure provide a T7 RNAP variant having increased RNA polymerase activity as compared to a naturally occurring T7 RNA polymerase.

RNA polymerase activity refers to the property of the T7 RNAP variant to synthesize RNA polymers. The activity of a T7 RNAP variant, for example, is assessed based on fidelity and polymerization kinetics (e.g., rate of polymerization). For example, one unit of a T7 RNAP variant may incorporate 10 nmoles of NTP into acid insoluble material (e.g., RNA product) in 30 minutes at a temperature of 37° C.

In some embodiments, the T7 RNAP variant may remain active (able to catalyze the polymerization reaction at a temperature of 37° C. or greater). In some embodiments, the T7 RNAP variant may remain active at a temperature of 42° C., or higher. In some embodiments, the T7 RNAP variant may remain active at a temperature of 42° C., 43° C., 44° C., 45° C., 46° C., 47° C., 48° C., 49° C., 50° C., 51° C., 52° C., 53° C., 54° C., 55° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

In some embodiments, the T7 RNAP variant may remain active at a temperature greater than 37° C. for 15 minutes to 48 hours, or longer. For example, the T7 RNAP variant may remain active at a temperature greater than 37° C. for 1, 2, 3, 4, 5, 6, 7, 8, 9, 10, 11, 12, 13, 14, 15, 16, 17, 18, 19, 20, 24, 36, 42, or 48 hours.

T7 RNAP variants described herein may remain active at an elevated temperature that denatures a control RNA polymerase. Thus, the T7 RNAP variant, in some embodiments, retains greater than 10% of its activity at an elevated temperature (e.g., above 37° C.) that would otherwise inactivate (i.e., less than 20%, less than 10%, less than 5%, less than 2%, less than 1%, or 0% of its original activity) a control RNA polymerase. In some embodiments, the T7 RNAP variant may retain 15%, 20%, 25%, 30%, 35%, 40%, 45%, 50%, 55%, 60%, 65%, 70%, 75%, 80%, 85%, 90%, 95%, 98%, 99%, or 100% activity at an elevated temperature (e.g., above 37° C.) that would otherwise inactivate a control RNA polymerase.

In some embodiments, the T7 RNAP variant may retain 10-100%, 25-100%, or 50-100% activity at an elevated temperature (e.g., above 37° C.) that would otherwise inactivate a control RNA polymerase. In some embodiments, the T7 RNAP variant may retain 10-90%, 10-85%, 10-80%, 10-75%, 10-70%, 10-65%, 10-60%, 10-55%, 25-90%, 25-85%, 25-80%, 25-75%, 25-70%, 25-65%, 25-60%, 25-55%, 50-90%, 50-85%, 50-80%, 50-75%, 50-70%, 50-65%, 50-60%, or 50-55% activity at an elevated temperature (e.g., above 37° C.) that would otherwise inactivate a control RNA polymerase.

Thus, the T7 RNAP variant, in some embodiments, may produce at least 10% more RNA product than a control RNA polymerase at a temperature of 37° C. or greater. In some embodiments, the T7 RNAP variant produces at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 100% more RNA product than a control RNA polymerase at a temperature greater than 37° C.

Some aspects of this disclosure provide a T7 RNAP variant having improved thermal stability as compared to a control T7 RNA polymerase, which is a naturally occurring T7 RNA polymerase. Thermal stability refers to the property of the T7 RNAP variant to resist denaturation at elevated temperatures. For example, a control T7 RNA polymerase may be partially or completely denatured (inactivated) at a temperature of 42° C., and a T7 RNAP variant is considered “thermostable” and does not denature at 42° C.

In some embodiments, the T7 RNAP variant has increased thermal stability (e.g., increased resistance to denaturation) at a temperature greater than 37° C., greater than 38° C., greater than 39° C., greater than 40° C., greater than 41° C., greater than 42° C., greater than 43° C., greater than 44° C., greater than 45° C., greater than 46° C., greater than 47° C., greater than 48° C., greater than 49° C., greater than 50° C., greater than 51° C., greater than 52° C., greater than 53° C., greater than 54° C., greater than 55° C., greater than 56° C., greater than 57° C., greater than 58° C., greater than 59° C., greater than 60° C., greater than 61° C., greater than 62 ° C., greater than 63° C., greater than 64° C., greater than 65° C., greater than 66° C., greater than 67° C., greater than 68° C., greater than 69° C., greater than 70° C., greater than 71° C., greater than 72° C., greater than 73° C., greater than 74° C., greater than 75° C., greater than 76° C., greater than 77° C., greater than 78° C., greater than 79° C., or greater than 80° C. as compared to the control RNA polymerase.

### Methods of Use

The present disclosure encompasses the use of a T7 RNAP variant in a variety of methods including, but not limited to, methods of producing RNA (e.g., in vitro transcription, in vivo transcription), methods of producing labeled RNA probes (e.g., radiolabeled RNA probes), methods for preparing a RNA vaccine, methods of polymerizing nucleotides, methods for amplifying RNA, and methods for producing proteins.

T7 RNAP variants described herein have increased thermal stability as compared to a control RNA polymerase, thus methods of use of the T7 RNAP variants described herein may be performed at a temperature greater than 37° C.

In some embodiments, the method of use of the T7 RNAP variant is performed at a temperature greater than 37° C., greater than 38° C., greater than 39° C., greater than 40° C., greater than 41° C., greater than 42° C., greater than 43° C., greater than 44° C., greater than 45° C., greater than 46° C., greater than 47° C., greater than 48° C., greater than 49° C., greater than 50° C., greater than 51° C., greater than 52° C., greater than 53° C., greater than 54° C., greater than 55° C., greater than 56° C., greater than 57° C., greater than 58° C., greater than 59° C., greater than 60° C., greater than 61° C., greater than 62° C., greater than 63° C., greater than 64° C., greater than 65° C., greater than 66° C., greater than 67° C., greater than 68° C., greater than 69° C., greater than 70° C., greater than 71° C., greater than 72° C., greater than 73° C., greater than 74° C., greater than 75° C., greater than 76° C., greater than 77° C., greater than 78° C., greater than 79° C., or greater than 80° C.

T7 RNAP variants disclosed herein provide certain advantages over T7 RNA polymerases, for example, for producing a RNA of interest at a temperature of 37° C. or greater. In some embodiments, a T7 RNAP variant produces a greater amount of a RNA of interest than a control RNA polymerase at a temperature of 37° C. or greater.

In some embodiments, the amount of RNA of interest produced by the T7 RNAP variant is at least 1.2-fold greater than an amount of the RNA of interest produced using a control RNA polymerase at a temperature of 37° C. or greater. In some embodiments, the amount of RNA of interest produced by the T7 RNAP variant is at least 1.3-fold greater, at least 1.4-fold greater, at least 1.5-fold greater, at least 1.6-fold greater, at least 1.7-fold greater, at least 1.8-fold greater, at least 1.9-fold greater, at least 2-fold greater, at least 2.5-fold greater, at least 3-fold greater, at least 4-fold greater, at least 5-fold greater, at least 6-fold greater, at least 7-fold greater, at least 8-fold greater, at least 9-fold greater, at least 10-fold greater, at least 11-fold greater, at least 12-fold greater, at least 13-fold greater, at least 14-fold greater, at least 15-fold greater, at least 20-fold greater, at least 25-fold greater than an amount of the RNA of interest produced using a control RNA polymerase at a temperature of 37° C. or greater.

Conditions suitable for the production of RNA (e.g., RNA product, labeled RNA, RNA vaccine, or amplified RNA) are known in the art or may be determined by one of ordinary skill in the art, taking into consideration, for example, optimal conditions for T7 RNA polymerase activity, including pH (e.g., pH 8), temperature (e.g., 15° C. to 70° C.), length of time (e.g., 5 min to 72 hrs), salt concentration (e.g., sodium chloride, potassium chloride, sodium acetate, and/or potassium acetate at a concentration of 5 mM to 1 M), and presence of phosphate and divalent ions (e.g. Mg²) of the reaction mixture as well as any exogenous cofactors.

In some embodiments, buffer is added to a reaction mixture, for example, to achieve a particular pH value and/or salt concentration. Examples of buffers include, without limitation, phosphate buffer, Tris buffer, MOPS buffer, HEPES buffer, citrate buffer, acetate buffer, malate buffer, MES buffer, histidine buffer, PIPES buffer, bis-tris buffer, and ethanolamine buffer.

In some embodiments, stability improving agents are added to a reaction mixture, for example, to improve activity and/or stability of various proteins. Non-limiting examples of stability improving agents include polyamines (e.g. spermidine, putrescine, cadaverine etc.), carrier proteins (e.g. BSA, etc.), pyrophosphatase, glycerol, diols (e.g. 1,2-propanediol, etc.), DMSO, salts (e.g. NaCl, MgCl₂, MnCl₂), reducing agents (e.g. Dithiothreitol (DTT), Tris (2-carboxyethyl) phosphine hydrochloride (TCEP), beta-mercaptoethanol).

In some embodiments, a reaction mixture during a RNA polymerization reaction is incubated for 0.5-24 hours at a temperature of 37° C., greater than 37° C., greater than 38° C., greater than 39° C., greater than 40° C., greater than 41° C., greater than 42° C., greater than 43 ° C., greater than 44° C., greater than 45° C., greater than 46° C., greater than 47° C., greater than 48° C., greater than 49° C., greater than 50° C., greater than 51° C., greater than 52° C., greater than 53° C., greater than 54° C., greater than 55° C., greater than 56° C., greater than 57° C., greater than 58° C., greater than 59° C., greater than 60° C., greater than 61° C., greater than 62° C., greater than 63° C., greater than 64° C., greater than 65° C., greater than 66° C., greater than 67° C., greater than 68° C., greater than 69° C., greater than 70° C., greater than 71° C., greater than 72° C., greater than 73° C., greater than 74° C., greater than 75° C., greater than 76° C., greater than 77° C., greater than 78° C., greater than 79° C., or greater than 80° C.

RNA produced by the methods provided herein may be any form of RNA, including single-stranded RNA (ssRNA) and double-stranded RNA (dsRNA). Non-limiting examples of single-stranded RNA include messenger RNA (mRNA), micro RNA (miRNA), small interfering RNA (siRNA), piwi-interacting RNA (piRNA), and antisense RNA. Double-stranded RNA herein includes wholly double-stranded molecules that do not contain a single-stranded region (e.g., a loop or overhang), as well as partially double-stranded molecules that contain a double-stranded region and a single-stranded region (e.g., a loop or overhang). Thus, short hairpin RNA (shRNA) may be produced by the methods of the present disclosure. In some embodiments, the RNA product binds to a target nucleic acid and may be used, for example, as a therapeutic, prophylactic, or diagnostic agent. In some embodiments, a RNA of interest is RNA of interest is dsRNA, ssRNA, siRNA, miRNA, piRNA, mRNA, shRNA or guide RNA (gRNA).

RNA produced by the methods provided herein may be modified as described herein. In some embodiments, RNA is produced according to a method described herein and subsequently modified. In some embodiments, RNA is produced according to a method described herein using a modified starting material. In some embodiments, the modified starting material is a modified nucleobase. In some embodiments, the modified starting material is a modified nucleoside. In some embodiments, the modified starting material is a modified nucleotide.

In some embodiments, modified RNA comprises a backbone modification. In some instances, backbone modification results in a longer half-life for the RNA due to reduced nuclease-mediated degradation. This is turn results in a longer half-life. Examples of suitable backbone modifications include, but are not limited to, phosphorothioate modifications, phosphorodithioate modifications, p-ethoxy modifications, methylphosphonate modifications, methylphosphorothioate modifications, alkyl- and aryl-phosphates (in which the charged phosphonate oxygen is replaced by an alkyl or aryl group), alkylphosphotriesters (in which the charged oxygen moiety is alkylated), peptide nucleic acid (PNA) backbone modifications, locked nucleic acid (LNA) backbone modifications, and the like. These modifications may be used in combination with each other and/or in combination with phosphodiester backbone linkages.

Alternatively or additionally, RNA may comprise other modifications, including modifications at the base or the sugar moieties. Examples include RNA having sugars which are covalently attached to low molecular weight organic groups other than a hydroxyl group at the 3′ position and other than a phosphate group at the 5′ position (e.g., a 2′-O-alkylated ribose), RNA having sugars such as arabinose instead of ribose. RNA also embrace substituted purines and pyrimidines such as C-5 propyne modified bases (Wagner et al., Nature Biotechnology 14:840-844, 1996). Other purines and pyrimidines include, but are not limited to, 5-methylcytosine, 2-aminopurine, 2-amino-6-chloropurine, 2,6-diaminopurine, and hypoxanthine. Other forms of modified RNA production may include use of modified nucleotides in the reaction mixture such as 5′-methyl-CTP, pseudouridine, 2′-O-methyl-UTP, 2-fluoro modified pyrimidines. Other such modifications are well known to those of skill in the art.

Any suitable DNA template encoding the RNA of interest may be used in the methods described herein. A DNA template includes a promoter, optionally an inducible promoter, operably linked to nucleotide sequence encoding a desired RNA product and, optionally, a transcriptional terminator. A DNA template is typically provided on a vector, such as a plasmid, although other template formats may be used (e.g., linear DNA templates generated by polymerase chain reaction (PCR), chemical synthesis, or other means known in the art). In some embodiments, more than one DNA template is used in a reaction mixture. In some embodiments, 2, 3, 4, 5, or more different DNA templates are used in a reaction mixture.

A promotor or terminator may be a naturally-occurring sequence or an engineered sequence. In some embodiments, an engineered sequence is modified to enhance transcriptional activity. In some embodiments, the promotor is a naturally-occurring sequence. In other embodiments, the promoter is an engineered sequence. In some embodiments, the terminator is a naturally-occurring sequence. In other embodiments, the terminator is an engineered sequence.

T7 RNAP variants in any suitable form may be used in the methods described herein. In some embodiments, the T7 RNAP variant is provided as a cell lysate from cells that express the T7 RNAP variant. In some embodiments, the T7 RNAP variant is provided as an enzyme preparation from cells that express the T7 RNAP variant. The enzyme preparation may be purified, partially purified, or unpurified. In some embodiments, the enzyme preparation comprises the T7 RNAP variant and cells or cellular components used to express the T7 RNAP variant. In some embodiments, the enzyme preparation comprises the T7 RNAP variant purified (e.g., essentially free) from cells or cellular components. In some embodiments, the T7 RNAP variant is provided by nucleic acids encoding the T7 RNAP variant.

### Kits

Any of the T7 RNAP variants described herein may be provided in a kit. In some embodiments, the kit comprises a T7 RNAP variant provided herein. In some embodiments, the kit comprises a nucleic acid vector for expressing a T7 RNAP variant as described herein.

In some embodiments, the kit further comprises at least one reagent for performing a method described herein including, but not limited to, methods of producing RNA, methods of labeled RNA probes, methods of preparing a RNA vaccine, methods of polymerizing nucleotides, and methods for amplifying RNA. In some embodiments, the at least one reagent includes, but is not limited to, a ribonucleoside triphosphate, a reaction buffer, and a DNA template.

The kit described herein may include one or more containers housing components for performing the methods described herein and optionally instructions of uses. Any of the kit described herein may further comprise components needed for performing the assay methods. Each component of the kits, where applicable, may be provided in liquid form (e.g., in solution), or in solid form, (e.g., a dry powder). In certain cases, some of the components may be reconstitutable or otherwise processible (e.g., to an active form), for example, by the addition of a suitable solvent or other species (e.g., water or buffer), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

### EXAMPLES

In order that the invention described herein may be more fully understood, the following examples are set forth. The examples described in this application are offered to illustrate the methods and compositions provided herein and are not to be construed in any way as limiting their scope.

### Example 1

### T7 RNA Polymerase V5ariants

The disclosure provides T7 RNA polymerase variants, for example, RNA polymerase proteins from one or more organisms, which comprise at least one amino acid substitution as described herein. In some embodiments, at least of the amino acid residues, identified below by a dot, of a RNA polymerase protein may be mutated. In some embodiments, the I320, I396, F546, S684, and G788 residues of the amino acid sequence provided in SEQ ID NO: 1, or a corresponding mutation in any of the amino acid sequences provided in SEQ ID NOs: 11-15, are mutated.

A number of RNA polymerase sequences from various species were aligned to demonstrate that corresponding homologous amino acid residues of I320, I396, F546, S684, and G788 of SEQ ID NO: 1 can be identified in other RNA polymerase proteins, allowing the generation of RNA polymerase variants with corresponding mutations of the homologous amino acid residues. The alignment was carried out using the NCBI Constraint-based Multiple Alignment Tool (COBALT(accessible at st-va.ncbi.nlm.nih.gov/tools/cobalt), with the following parameters. Alignment parameters: Gap penalties -11,-1; End-Gap penalties -5,-1. CDD Parameters: Use RPS BLAST on; Blast E-value 0.003; Find Conserved columns and Recompute on. Query Clustering Parameters: Use query clusters on; Word Size 4; Max cluster distance 0.8; Alphabet Regular.

An exemplary alignment of four RNA polymerase sequences is provided below. The RNA polymerase sequences in the alignment are: (T7): Wild-type T7 RNA polymerase from Bacteriophage T7, GenBank: FJ881694.1, SEQ ID NO: 1; (T3): RNA polymerase from Bacteriophage T3, NCBI Reference Sequence: NP_523301.1, SEQ ID NO: 11; (SP6) RNA polymerase from Bacteriophage SP6, UniProt: P06221, SEQ ID NO: 12; and (FE44) RNA Polymerase from Erwinia phage FE44, NCBI Reference Sequence: YP_008766719.1, SEQ ID NO: 13. Amino acid residues I320, I396, F546, S684, and G788 in wild-type T7 RNA polymerase and the homologous amino acids in the aligned sequences are identified with a dot above the amino acid residues.

| | | | |
|---|---|---|---|
| T7 | 1 | M-NTI-NIAKNDFSDIELAAIPFNTLADHYGERLAREQLALEHESYEMGEARFRKMFERQLKAGEVADNAAAKPLITTLL | 78 |
| T3 | 1 | M-NIIENIEKNDFSEIELAAIPFNTLADHYGSALAKEQLALEHESYELGERRFLKMLERQAKAGEIADNAAAKPLLATLL | 79 |
| SP6 | 1 | MQDLH------------------------------AIQTQLEEENFNGGIRRFEADQQRQIAAGSESDTAWNRRLLSELI | 50 |
| FE44 | 1 | MTNVI-NAPKNDFSDIANAIQPYNILADHYGAHLAATQLELEHEAHTEGEKRFLKAMERQIKAGEFGDNAVAKPLLSSLA | 79 |
| T7 | 79 | PKMIARINDWFEEVKAKRGKRPTAFQFLQEIKPEAVAYITIKTTLACLTSADNTTVQAVASAIGRAIEDEARFGRIRDLE | 158 |
| T3 | 80 | PKLTTRIVEWLEEYASKKGRKPSAYAPLQLLKPEASAFITLKVILASLTSTNMTTIQAAAGMLGKAIEDEARFGRIRDLE | 159 |
| SP6 | 51 | APMAEGIQAYKEEYEGKKGRAPRALAFLQCVENEVAAYITMKVVMDMLNTD--ATLQATAMSVAERIEDQVRFSKLEGHA | 128 |
| FE44 | 80 | PKFIEAWNTWFTEVESKRGKRPVAYNLVQKVAPEAAAFITLKVTLACLTKEEYTNLQSVATKIGRSIEDELRFGRIRDEE | 159 |
| T7 | 159 | AKHFKKNVEEQLNKRVGHVYKKAF-MQVVEADMLSKGLLGGEAWSSWHKEDSIHVGVRCIEMLIEST---GMVSLHRQNA | 234 |
| T3 | 160 | AKHFKKHVEEQLNKRHGQVYKKAF-MQVVEADMIGRGLLGGEAWSSWDKETTMHVGIRLIEMLIEST---GLVELQRHNA | 235 |
| SP6 | 129 | AKYFEK-VKKSLKASRTKSYRHAHNVAVVAEKSVAEKDADFDRWEAWPKETQLQIGTTLLEILEGSVFYNGEPVFMRAMR | 207 |
| FE44 | 160 | AKHFKNHVQEALNKRVGIVYKKAF-MQAVEGKMLDAGQLQTK-WTTWTPEESIHVGVRMLELLIGST---GLVELHRPFA | 234 |
| T7 | 235 | GVVGQDSETIELAPEYAEAFATRAGALAGISPMFQPCVVPPKPWTGITGGGYWANGRRPLALVRTHSKKALMRYEDVYMP | 314 |
| T3 | 236 | GNAGSDHEALQLAQEYVDVLAKRAGALAGISPMFQPCVVPPKPWVAITGGGYWANGRRPLALVRTHSKKGLMRYEDVYMP | 315 |
| SP6 | 208 | TYGGKTIYYLQTSESVGQWISAFKEHVAQLSPAYAPCVIPPRPWRTPFNGGFHTEKVASRIRLVKGNREHVRKLTQKQMP | 287 |
| FE44 | 235 | GNVEKDGEYIQLTEQYVDLLSKRAGALAAIAPMYQPCVVPPKPWTSPVGGGYWAAGRKPLSLVRTGSKKGLERYNDVYMP | 314 |
| | | • | |
| T7 | 315 | EVYKAINIAQNTAWKINKKVLAVANVITKWK--HCPVEDIPAIEREELPMKPEDIDMNPEALTA---------------W | 377 |
| T3 | 316 | EVYKAVNLAQNTAWKINKKVLAVVNEIVNWK--NCPVADIPSLERQELPPKPDDIDTNEAALKE---------------W | 378 |
| SP6 | 288 | KVYKAINALQNTQWQINKDVLAVIEEVIRLDLGYGVPSFKPLIDKENKPANPVPVEFQHLRGRELKEMLSPEQWQQFINW | 367 |
| FE44 | 315 | EVYKAVNIAQNTPWKINKKVLAVVNEIVNWK--HCPVDDVPALERGELPVKPEDIDTNEVALKA---------------W | 377 |
| | | • | |
| T7 | 378 | KRAAAAVYRKDKARKSRRISLEFMLEQANKFANHKAIWFPYNMDWRGRVYAVSM-FNPQGNDMTKGLLTLAKGKPI-GKE | 455 |
| T3 | 379 | KKAAAGIYRLDKARVSRRISLEFMLEQANKFASKKAIWFPYNMDWRGRVYAVPM-FNPQGNDMTKGLLTLAKGKPI-GEE | 456 |
| SP6 | 368 | KGECARLYTAETKRGSKSAAVVRMVGQARKYSAFESIYFVYAMDSRSRVYVQSSTLSPQSNDLGKALLRFTEGRPVNGVE | 447 |
| FE44 | 378 | KKAASAIYRKEKARVSRRMSMEFMLGQANKFAQFKAIWFPMNMDWRGRVYAVPM-FNPQGNDMTKGLLTLAKGKPI-GVD | 455 |
| T7 | 456 | GYYWLKIHGANCAGVDKVPFPERIK--FIEENHENIMACAKSPLENTWWAEQDSPFCFLAFCFEYAGVQHHG-----LSY | 528 |
| T3 | 457 | GFYWLKIHGANCAGVDKVPFPERIA--FIEKHVDDILACAKDPINNTWWAEQDSPFCFLAFCFEYAGVTHHG-----LSY | 529 |
| SP6 | 448 | ALKWFCINGANLWGWDKKTFDVRVSNVLDEEFQDMCRDIAADPLTFTQWAKADAPYEFLAWCFEYAQYLDLVDEGRADEF | 527 |
| FE44 | 456 | GYYWLKIHGANTAGVDKVDFAERIK--FIEDNHENIMSVAADPIANTWWAEQDSPFCFLAFCFEYAGVQHHG-----MNY | 528 |
| | | • | |
| T7 | 529 | NCSLPLAFDGSCSGIQHFSAMLRDEVGGRAVNLLPSETVQDIYGIVAKKVNEILQADAINGTDNEVVTVTDENTGEISEK | 608 |
| T3 | 530 | NCSLPLAFDGSCSGIQHFSAMLRDEVGGRAVNLLPSETVQDIYGIVAQKVNEILKQDAINGTPNEMITVTDKDTGEISEK | 609 |
| SP6 | 528 | RTHLPVHQDGSCSGIQHYSAMLRDEVGAKAVNLKPSDAPQDIYGAVAQVVIKKNALYMDADD------ATTFTSGSVTLS | 601 |
| FE44 | 529 | NCSLPLAFDGSCSGIQHFSAMLRDEIGGRAVNLLPSKEVQDIYRIVAERVNEILKQDVINGTDNEVETVTNKDTGEITEK | 608 |
| | | • | |
| T7 | 609 | VKLGTKALAGQWLAYGVTRSVTKRSVMTLAYGSKEFGFRQQVLEDTIQPAIDSGKGLMFTQPNQAAGYMAKLIWESVSVT | 688 |
| T3 | 610 | LKLGTSTLAQQWLAYGVTRSVTKRSVMTLAYGSKEFGFRQQVLDDTIQPAIDSGKGLMFTQPNQAAGYMAKLIWDAVSVT | 689 |
| SP6 | 602 | -GTELRAMASAWDSIGITRSLTKKPVMTLPYGSTRLTCRESVIDYIVDLEEKEAQKAVAE----GRTANKVHPFEDDRQD | 676 |
| FE44 | 609 | LKLGTKELAGQWLAYGVTRKVTKRSVMTLAYGSKEYGFRDQVLEDTIQPAIDDGKGLMFTQPNQAAGYMAKLIWNAVTVT | 688 |
| T7 | 689 | VVAAVEAMNWLKSAAKLLAAEVKD------KKTGEILR----KRCAVHWVTPDGFPVWQEYKKPIQTRLNLMFLGQFRLQ | 758 |
| T3 | 690 | VVAAVEAMNWLKSAAKLLAAEVKD------KKTKEILR----HRCAVHWTTPDGFPVWQEYRKPLQKRLDMIFLGQFRLQ | 759 |
| SP6 | 677 | YLTPGAAYNYMTALIWPSISEVVKAPIVAMKMIRQLARFAAKRNEGLMYTLPTGFILEQKIMATEMLRVRTCLMGDIKMS | 756 |
| FE44 | 689 | VVAAVEAMNWLKSAAKLLAAEVKD------KKTKEVLR----KRCAVHWVTPDGFPVWQEYRKPVQTRLNLMFLGQIRLQ | 758 |
| | | • | |
| T7 | 759 | PTINTNKDSEIDAHKQESGIAPNFVHSQDGSHLRKTVVWAHEKYGIESFALIHDSFGTIPADAANLFKAVRETMVDTYES | 838 |
| T3 | 760 | PTINTLKDSGIDAHKQESGIAPNFVHSQDGSHLRMTVVYAHEKYGIESFALIHDSFGTIPADAGKLFKAVRETMVITYEN | 839 |
| SP6 | 757 | LQVET---DIVDEAAMMGAAAPNFVHGHDASHLILTVCELVDK-GVTSIAVIHDSFGTHADNTLTLRVALKGQMVAMYID | 832 |
| FE44 | 759 | PTVNTNKDSGIDARKQESGIAPNFVHSMDGSHLRMTVVRSNEVYGVESFALIHDSFGTIPADAGNLFKAVRETMVNTYEE | 838 |
| T7 | 839 | CDVLADFYDQFADQLHESQLDKMPALPAKGNLNLRDILESDFAFA (SEQ ID NO: 1) | 883 |
| T3 | 840 | NDVLADFYSQFADQLHETQLDKMPPLPKKGNLNLQDILKSDFAFA (SEQ ID NO: 11) | 884 |
| SP6 | 833 | GNALQKLLEEHEVRWMVDTG---IEVPEQGEFDLNEIMDSEYVFA (SEQ ID NO: 12) | 874 |
| FE44 | 839 | NDVLADFYDQFADQLHESQLDKMPEMPAKGSLDLQEILKSDFAFA (SEQ ID NO: 13) | 883 |

The alignment demonstrates that amino acid sequences and amino acid residues that are homologous to a T7 RNA polymerase amino acid sequence or amino acid residue can be identified across RNA polymerase sequences, including but not limited to RNA polymerase sequences from different species, by identifying the amino acid sequence or residue that aligns with the T7 RNA polymerase amino acid sequence or the T7 RNA polymerase residue using alignment programs and algorithms known in the art.

This disclosure provides RNA polymerase variants in which one or more of the amino acid residues identified by a dot in SEQ ID NOs: 11-13 (e.g., T3, SP6, and FE44, respectively) are mutated as described herein. The residues I320, I396, F546, S684, and G788 in T7 RNA polymerase of SEQ ID NO: 1 that correspond to the residues identified in SEQ ID NOs: 11-13 by a dot are referred to herein as “homologous” or “corresponding” residues. Such homologous residues can be identified by sequence alignment, e.g., as described above, and by identifying the sequence or residue that aligns with the T7 RNA polymerase sequence or residue. Similarly, mutations in T7 RNA polymerase sequences that correspond to mutations identified in SEQ ID NO: 1 herein, e.g., mutations of residues I320, I396, F546, S684, and G788 in SEQ ID NO: 1, are referred to herein as “homologous” or “corresponding” mutations. For example, the amino acid substitution corresponding to the amino acid substitution at position I320 in SEQ ID NO: 1 for the aligned sequences above are V321 for T3, I293for SP6, and V320 for FE44.

RNA polymerase sequences from different species are known in the art. Amino acid residues corresponding to residues I320, I396, F546, S684, and G788 in T7 RNA polymerase of SEQ ID NO: 1 may be identified as described herein for RNA polymerase sequences known in the art. Any of the identified RNA polymerase sequences may be used in accordance with the present disclosure.

### Example 2

### Cell-Free Synthesis of RNA Using a Wild-Type T7 RNA Polymerase or a Thermostable T7 RNA Polymerase Variant

### Materials and Methods

### Cloning of Variant Polymerases

Mutations were created via site directed mutagenesis using primers. Mutations in the T7 RNA polymerase variants described herein are shown in Table 1. A variant version of the polymerase was generated via overlap PCR reactions using specific primers and native T7 RNA polymerase (Uniprot P00573) as the template. Furthermore, six histidine residues were introduced into these variant polymerases at the N-terminus to facilitate with protein purification during the PCR step via custom primers. The obtained PCR product was cloned into a vector pBAD24 using Nhel and HindIII restriction enzymes. Such a process was carried out for each individual mutation. This process was repeated using a pre-existing variant as the PCR template to generate more than one mutation. Each variant polymerase was sequenced to ensure accuracy at DNA level as well as at the protein level after purification. The wild type T7 RNA polymerase was also his-tagged and cloned in pBAD24 similarly to serve as a control for native activity.

TABLE 1
Mutant T7 RNA polymerase variants.

| Mutant # | Mutations |
|---|---|
| M1 | Ile320Leu, Ile396Leu, Phe546Trp, Ser684Ala, Gly788Ala |
| M2 | Ile320Leu, Ile396Leu, Gly788Ala |
| M3 | Ile396Leu, Ser684Ala, Gly788Ala |
| M4 | Ile320Leu, Ser684Ala, Gly788Ala |

### Protein Expression and Cell Growth

The plasmids carrying different his-tagged variant polymerase sequences were transformed into E. coli BL21 strain lacking the chromosomal T7 RNAP to generate host strains for protein expression. The transformed E. coli strains were grown using 1% inoculum into Luria Broth (with carbenicillin antibiotic) at 37° C. with a constant agitation of 250 rpm. Cultures were induced with 0.2% Arabinose for 4 hours when the OD₆₀₀ reached 0.6. At the end of 4 hours of induction, cultures were harvested via centrifugation and the collected biomass was kept frozen at −80° C. for future testing. Samples were collected before induction and at the end of study for SDS-PAGE analysis (to verify protein expression) and for OD₆₀₀ determination.

Medium, Chemicals and Buffers Luria Broth (Sigma Aldrich) was used for cell growth (10 g/L Tryptone, 5 g/L yeast extract and 5 g/L NaCl). For protein purification 1× Wash buffer (20 mM Sodium phosphate, 500 mM NaCl, 40 mM Imidazole, at pH 7.4) was used for resuspending biomass, for column equilibration and for washing the column. The composition of elution buffer used is as follows: 20 mM Sodium phosphate, 500 mM NaCl, 750 mM Imidazole, at pH 7.4. The composition of 2× dialysis buffer used is as follows: 2×PBS, 5 mM DTT, 0.01% Triton X 100.

For thermostability/activity testing the following commercially available chemicals/reagents were used: Ribonucleotide solution (ATP/GTP/CTP/UTP); Spermidine; MgSO₄, Thermostable inorganic pyrophosphatase (TIPP). The composition of 10× reaction buffer (10×RB) is as follows: 300 mM MgSO₄, 20 mM Spermidine. The quench buffer composition is as follows: 20 mM Tris-HCl pH 8.0, 10 mM EDTA pH 8. After quenching the reactions, DNase I was used to remove the background DNA template. RNA loading dye (2×) was used while running agarose gels. DNA template used was a linearized plasmid carrying the coding sequence (524 bp).

### Generation of Crude Lysates and Protein Purification

To purify a protein of interest, first the biomass was lysed, and the generated crude lysate was clarified and used to purify the target protein of interest. To generate a crude lysate, the biomass was first resuspended in 1X Wash buffer and then lysed using high pressure homogenization. Following lysis, the crude lysates were centrifuged for 60 minutes at 15,000g, 4° C. After centrifugation the supernatant (clarified lysate) was decanted and used for purification.

The clarified lysates were used for Ni ion affinity chromatography-based purification using FPLC (AKTA prime plus) using ion exchange/gradient elution protocol. 1 mL his-columns were procured from GE Healthcare. All buffers used are listed above. Following purification, the proteins were dialyzed overnight for 16 hours at the end of which the proteins were stocked in −20° C. using 50% glycerol (final concentration).

### Activity and Thermostability Testing

Activity testing of the mutant polymerases were performed via in vitro transcription (IVT) reactions using the IVT reaction mixture (described herein). Activity testing of the candidate mutant polymerases were performed between 37° C.-54° C. The reactions were set-up at 25 μL scale in PCR plates and incubated for two hours at the test temperature using a thermal cycler. Following this, the reactions were quenched and processed for downstream analysis. The IVT reaction mixture is described in Table 2.

TABLE 2
IVT reaction mixture composition (25 μL).

| IVT reaction mixture | Final concentration |
|---|---|
| Nuclease-free water | N/A |
| NTPs mixture (mM) | 4 |
| Reaction buffer (MgSO₄/Spermidine) (mM) | (30/2) |
| DNA Template (ng/μL) | 100 |
| T7 RNAP candidate (mg/mL) | 0.012 |
| Pyrophosphate (TIPP) (2000 U/mL stock) | 40 |

To quench the reaction after 2 hours, 2.5 μL of DNase I was added to the reaction and incubated at 37° C. for 30 mins to remove the background template. Next, 22.5 μL of water was added to the mixture. This mixture serves as the quenched reaction mixture and was used for downstream analysis for RNA product verification/quantification via agarose gel electrophoresis or via HPLC.

For agarose gel electrophoresis, 5 μL was removed from the quenched reaction mixture and added to 10 μL of quench buffer and mixed well in a PCR plate. Next, 15 μL of 2× RNA dye was added and this mixture was heated at 70° C. for 10 mins in a thermalcycler.

About, 10 μL of this mixture was then run on a 2% agarose gel stained with SYBR safe for 60 mins at 140 V before imaging.

The RNA synthesized in the reaction was purified via an adapted RNASwift extraction protocol and quantitated using a reverse phase ion pair chromatography as described (Nwokeji, A. O., Kilby, P. M., Portwood, D. E., & Dickman, M. J. (2016). RNASwift: A rapid, versatile RNA extraction method free from phenol and chloroform. Analytical Biochemistry, 512, 36 -46).

### HPLC Analysis of Purified Product

To quantitate dsRNA, a solid phase extraction protocol was performed to remove unwanted proteins. The primary separating mechanism was reverse phase ion pair chromatography. Signal was measured at 260 nm using a diode array detector. By normalizing areas to the internal standard response, results were corrected for any losses that may have occurred in the extraction process and concentrations were calculated based slopes generated from external calibration curves.

### Results

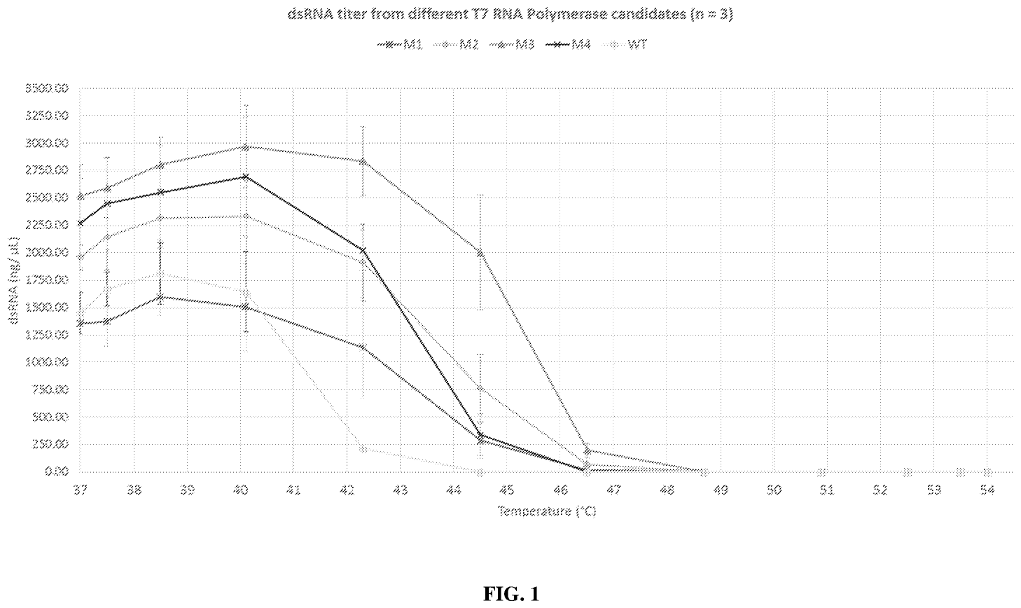

To test the activity of mutant T7 RNA polymerases, an IVT reaction was set up for 2 hours as described herein and the amount of RNA produced was quantified via HPLC. The wild type T7 RNA polymerase served as reference control for native T7 RNA polymerase activity. A negative control was also included, where T7 RNA polymerase was replaced with water. No dsRNA product was detected from the negative control reactions (FIG. 1).

As shown in FIG. 1, M3 makes most dsRNA from 37° C.-42.3° C.; followed by M4 up to 40.1° C.; followed by M2 up to 38.5° C. M2, M3 and M4 T7 RNA polymerases make more dsRNA relative to the wild-type at temperatures ranging from 37° C.-46.5° C. While, M1 T7 RNA polymerase makes more dsRNA relative to the wild-type at temperatures ranging from 42.3° C.-46.5° C. Table 3 indicates the fold increase in dsRNA titer relative to the wild type polymerase. Table 4 indicates the average dsRNA titer (ng/μL) of mutant and wild-type T7 RNA polymerases.

TABLE 3
Fold increase in dsRNA titer relative to WT by
M1, M2, M3 and M4 T7 RNA polymerases.

| Temperature | | | | |
|---|---|---|---|---|
| (° C.) | M1 | M2 | M3 | M4 |
| 37 | 0.9 | 1.4 | 1.7 | 1.6 |
| 37.5 | 0.8 | 1.3 | 1.6 | 1.5 |
| 38.5 | 0.9 | 1.3 | 1.5 | 1.4 |
| 40.1 | 0.9 | 1.4 | 1.8 | 1.6 |
| 42.3 | 5.3 | 9.0 | 13.3 | 9.5 |
| 44.5 | WT not | WT not | WT not | WT not |
| | active | active | active | active |
| 46.5 | WT not | WT not | WT not | M4 not |
| | active | active | active | active |

TABLE 4
Average dsRNA titer (ng/μL) from mutant and wild-type
T7 RNA Polymerases after 2 hours of incubation at
reaction temperature in a 25 μL system (n = 3).

| Temperature | dsRNA titer (ng/μL) | | | | |
|---|---|---|---|---|---|
| (° C.) | M1 | M2 | M3 | M4 | WT |
| 37 | 1353 | 1958 | 2517 | 2269 | 1449 |
| 37.5 | 1373 | 2145 | 2590 | 2447 | 1668 |
| 38.5 | 1597 | 2313 | 2802 | 2549 | 1809 |
| 40.1 | 1505 | 2334 | 2969 | 2693 | 1644 |
| 42.3 | 1133 | 1911 | 2835 | 2022 | 212 |
| 44.5 | 285 | 760 | 2002 | 339 | 0.00 |
| 46.5 | 16 | 70 | 196 | 0.00 | 0.00 |
| 48.7 | 0.00 | 0.00 | 0.00 | 0.00 | 0.00 |
| 50.9 | 0.00 | 0.00 | 0.00 | 0.00 | 0.00 |
| 52.5 | 0.00 | 0.00 | 0.00 | 0.00 | 0.00 |
| 53.5 | 0.00 | 0.00 | 0.00 | 0.00 | 0.00 |
| 54 | 0.00 | 0.00 | 0.00 | 0.00 | 0.00 |

It was determined that WT retains 90% of its activity up to 40.1° C. and has an activity of only 12% relative to its maximum at 42.3° C. WT polymerase loses activity at 44.5° C. and higher. The fold increase in dsRNA titer relative to the control wild type is represented in Table 3. It is seen that M1 has a 5.3-fold increase in titer relative to the wild type at 42.3° C. M2, M3 and M4 make greater amount of RNA at all tested temperatures relative to the wild type polymerase. At 42.3° C. M2, M3, M4 make 8.9, 13.3 and 9.5 fold greater amount of RNA than the wild type protein.

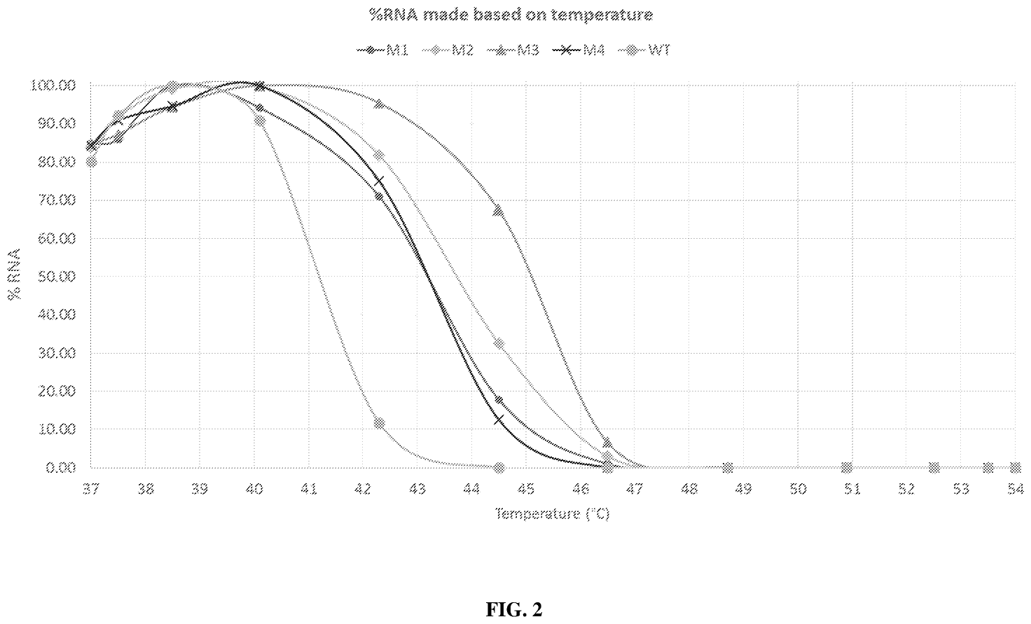

As shown in FIG. 2, M1-M4 have improved thermostability relative to WT. Thermostability of candidate T7 RNA polymerases is inferred as follows: M3>M2>M1>M4>WT. It can be seen that the wild type polymerases makes about 12% of RNA (212.42 ng/μL) at 42.3° C. relative to its maximum and does not make any RNA at 44.5° C. or higher relative to its maximum at 38.5° C. (1808.9 ng/μL).

Sequences

Wild-Type T7 RNA Polymerase from Bacteriophage T7
(GenBank: FJ881694.1/ Uniprot P00573)
MNTINIAKNDFSDIELAAIPFNTLADHYGERLAREQLALEHESYEMGEARFRKMFERQLKAG
EVADNAAAKPLITTLLPKMIARINDWFEEVKAKRGKRPTAFQFLQEIKPEAVAYITIKTTLA
CLTSADNTTVQAVASAIGRAIEDEARFGRIRDLEAKHFKKNVEEQLNKRVGHVYKKAFMQVV
EADMLSKGLLGGEAWSSWHKEDSIHVGVRCIEMLIESTGMVSLHRQNAGVVGQDSETIELAP
EYAEAIATRAGALAGISPMFQPCVVPPKPWTGITGGGYWANGRRPLALVRTHSKKALMRYED
VYMPEVYKAINIAQNTAWKINKKVLAVANVITKWKHCPVEDIPAIEREELPMKPEDIDMNPE
ALTAWKRAAAAVYRKDKARKSRRISLEFMLEQANKFANHKAIWFPYNMDWRGRVYAVSMFNP
QGNDMTKGLLTLAKGKPIGKEGYYWLKIHGANCAGVDKVPFPERIKFIEENHENIMACAKSP
LENTWWAEQDSPFCFLAFCFEYAGVQHHGLSYNCSLPLAFDGSCSGIQHFSAMLRDEVGGRA
VNLLPSETVQDIYGIVAKKVNEILQADAINGTDNEVVTVTDENTGEISEKVKLGTKALAGQW
LAYGVTRSVTKRSVMTLAYGSKEFGFRQQVLEDTIQPAIDSGKGLMFTQPNQAAGYMAKLIW
ESVSVTVVAAVEAMNWLKSAAKLLAAEVKDKKTGEILRKRCAVHWVTPDGFPVWQEYKKPIQ
TRLNLMFLGQFRLQPTINTNKDSEIDAHKQESGIAPNFVHSQDGSHLRKTVVWAHEKYGIES
FALIHDSFGTIPADAANLFKAVRETMVDTYESCDVLADFYDQFADQLHESQLDKMPALPAKG
NLNLRDILESDFAFA (SEQ ID NO: 1)
Engineered T7 RNA Polymerase (I320L I396L F546W S684A G788A)(Variant 1)
MNTINIAKNDFSDIELAAIPFNTLADHYGERLAREQLALEHESYEMGEARFRKMFERQLKAG
EVADNAAAKPLITTLLPKMIARINDWFEEVKAKRGKRPTAFQFLQEIKPEAVAYITIKTTLA
CLTSADNTTVQAVASAIGRAIEDEARFGRIRDLEAKHFKKNVEEQLNKRVGHVYKKAFMQVV
EADMLSKGLLGGEAWSSWHKEDSIHVGVRCIEMLIESTGMVSLHRQNAGVVGQDSETIELAP
EYAEAIATRAGALAGISPMFQPCVVPPKPWTGITGGGYWANGRRPLALVRTHSKKALMRYED
VYMPEVYKALNIAQNTAWKINKKVLAVANVITKWKHCPVEDIPAIEREELPMKPEDIDMNPE
ALTAWKRAAAAVYRKDKARKSRRLSLEFMLEQANKFANHKAIWFPYNMDWRGRVYAVSMFNP
QGNDMTKGLLTLAKGKPIGKEGYYWLKIHGANCAGVDKVPFPERIKFIEENHENIMACAKSP
LENTWWAEQDSPFCFLAFCFEYAGVQHHGLSYNCSLPLAFDGSCSGIQHWSAMLRDEVGGRA
VNLLPSETVQDIYGIVAKKVNEILQADAINGTDNEVVTVTDENTGEISEKVKLGTKALAGQW
LAYGVTRSVTKRSVMTLAYGSKEFGFRQQVLEDTIQPAIDSGKGLMFTQPNQAAGYMAKLIW
EAVSVTVVAAVEAMNWLKSAAKLLAAEVKDKKTGEILRKRCAVHWVTPDGFPVWQEYKKPIQ
TRLNLMFLGQFRLQPTINTNKDSEIDAHKQESGIAPNFVHSQDASHLRKTVVWAHEKYGIES
FALIHDSFGTIPADAANLFKAVRETMVDTYESCDVLADFYDQFADQLHESQLDKMPALPAKG
NLNLRDILESDFAFA(SEQ ID NO: 2)
Engineered T7 RNA Polymerase (I320L I396L G788A)(Variant 2)
MNTINIAKNDFSDIELAAIPFNTLADHYGERLAREQLALEHESYEMGEARFRKMFERQLKAG
EVADNAAAKPLITTLLPKMIARINDWFEEVKAKRGKRPTAFQFLQEIKPEAVAYITIKTTLA
CLTSADNTTVQAVASAIGRAIEDEARFGRIRDLEAKHFKKNVEEQLNKRVGHVYKKAFMQVV
EADMLSKGLLGGEAWSSWHKEDSIHVGVRCIEMLIESTGMVSLHRQNAGVVGQDSETIELAP
EYAEAIATRAGALAGISPMFQPCVVPPKPWTGITGGGYWANGRRPLALVRTHSKKALMRYED
VYMPEVYKALNIAQNTAWKINKKVLAVANVITKWKHCPVEDIPAIEREELPMKPEDIDMNPE
ALTAWKRAAAAVYRKDKARKSRRLSLEFMLEQANKFANHKAIWFPYNMDWRGRVYAVSMFNP
QGNDMTKGLLTLAKGKPIGKEGYYWLKIHGANCAGVDKVPFPERIKFIEENHENIMACAKSP
LENTWWAEQDSPFCFLAFCFEYAGVQHHGLSYNCSLPLAFDGSCSGIQHFSAMLRDEVGGRA
VNLLPSETVQDIYGIVAKKVNEILQADAINGTDNEVVTVTDENTGEISEKVKLGTKALAGQW
LAYGVTRSVTKRSVMTLAYGSKEFGFRQQVLEDTIQPAIDSGKGLMFTQPNQAAGYMAKLIW
ESVSVTVVAAVEAMNWLKSAAKLLAAEVKDKKTGEILRKRCAVHWVTPDGFPVWQEYKKPIQ
TRLNLMFLGQFRLQPTINTNKDSEIDAHKQESGIAPNFVHSQDASHLRKTVVWAHEKYGIES
FALIHDSFGTIPADAANLFKAVRETMVDTYESCDVLADFYDQFADQLHESQLDKMPALPAKG
NLNLRDILESDFAFA (SEQ ID NO: 3)
Engineered T7 RNA Polymerase (I396L S684A G788A)(Variant 3)
MNTINIAKNDFSDIELAAIPFNTLADHYGERLAREQLALEHESYEMGEARFRKMFERQLKAG
EVADNAAAKPLITTLLPKMIARINDWFEEVKAKRGKRPTAFQFLQEIKPEAVAYITIKTTLA
CLTSADNTTVQAVASAIGRAIEDEARFGRIRDLEAKHFKKNVEEQLNKRVGHVYKKAFMQVV
EADMLSKGLLGGEAWSSWHKEDSIHVGVRCIEMLIESTGMVSLHRQNAGVVGQDSETIELAP
EYAEAIATRAGALAGISPMFQPCVVPPKPWTGITGGGYWANGRRPLALVRTHSKKALMRYED
VYMPEVYKAINIAQNTAWKINKKVLAVANVITKWKHCPVEDIPAIEREELPMKPEDIDMNPE
ALTAWKRAAAAVYRKDKARKSRRLSLEFMLEQANKFANHKAIWFPYNMDWRGRVYAVSMFNP
QGNDMTKGLLTLAKGKPIGKEGYYWLKIHGANCAGVDKVPFPERIKFIEENHENIMACAKSP
LENTWWAEQDSPFCFLAFCFEYAGVQHHGLSYNCSLPLAFDGSCSGIQHFSAMLRDEVGGRA
VNLLPSETVQDIYGIVAKKVNEILQADAINGTDNEVVTVTDENTGEISEKVKLGTKALAGQW
LAYGVTRSVTKRSVMTLAYGSKEFGFRQQVLEDTIQPAIDSGKGLMFTQPNQAAGYMAKLIW
EAVSVTVVAAVEAMNWLKSAAKLLAAEVKDKKTGEILRKRCAVHWVTPDGFPVWQEYKKPIQ
TRLNLMFLGQFRLQPTINTNKDSEIDAHKQESGIAPNFVHSQDASHLRKTVVWAHEKYGIES
FALIHDSFGTIPADAANLFKAVRETMVDTYESCDVLADFYDQFADQLHESQLDKMPALPAKG
NLNLRDILESDFAFA (SEQ ID NO: 4)
Engineered T7 RNA Polymerase (I320L S684A G788A)(Variant 4)
MNTINIAKNDFSDIELAAIPFNTLADHYGERLAREQLALEHESYEMGEARFRKMFERQLKAG
EVADNAAAKPLITTLLPKMIARINDWFEEVKAKRGKRPTAFQFLQEIKPEAVAYITIKTTLA
CLTSADNTTVQAVASAIGRAIEDEARFGRIRDLEAKHFKKNVEEQLNKRVGHVYKKAFMQVV
EADMLSKGLLGGEAWSSWHKEDSIHVGVRCIEMLIESTGMVSLHRQNAGVVGQDSETIELAP
EYAEAIATRAGALAGISPMFQPCVVPPKPWTGITGGGYWANGRRPLALVRTHSKKALMRYED
VYMPEVYKALNIAQNTAWKINKKVLAVANVITKWKHCPVEDIPAIEREELPMKPEDIDMNPE
ALTAWKRAAAAVYRKDKARKSRRISLEFMLEQANKFANHKAIWFPYNMDWRGRVYAVSMFNP
QGNDMTKGLLTLAKGKPIGKEGYYWLKIHGANCAGVDKVPFPERIKFIEENHENIMACAKSP
LENTWWAEQDSPFCFLAFCFEYAGVQHHGLSYNCSLPLAFDGSCSGIQHFSAMLRDEVGGRA
VNLLPSETVQDIYGIVAKKVNEILQADAINGTDNEVVTVTDENTGEISEKVKLGTKALAGQW
LAYGVTRSVTKRSVMTLAYGSKEFGFRQQVLEDTIQPAIDSGKGLMFTQPNQAAGYMAKLIW
EAVSVTVVAAVEAMNWLKSAAKLLAAEVKDKKTGEILRKRCAVHWVTPDGFPVWQEYKKPIQ
TRLNLMFLGQFRLQPTINTNKDSEIDAHKQESGIAPNFVHSQDASHLRKTVVWAHEKYGIES
FALIHDSFGTIPADAANLFKAVRETMVDTYESCDVLADFYDQFADQLHESQLDKMPALPAKG
NLNLRDILESDFAFA (SEQ ID NO: 5)
Engineered T7 RNA Polymerase (I320L)
MNTINIAKNDFSDIELAAIPFNTLADHYGERLAREQLALEHESYEMGEARFRKMFERQLKAG
EVADNAAAKPLITTLLPKMIARINDWFEEVKAKRGKRPTAFQFLQEIKPEAVAYITIKTTLA
CLTSADNTTVQAVASAIGRAIEDEARFGRIRDLEAKHFKKNVEEQLNKRVGHVYKKAFMQVV
EADMLSKGLLGGEAWSSWHKEDSIHVGVRCIEMLIESTGMVSLHRQNAGVVGQDSETIELAP
EYAEAIATRAGALAGISPMFQPCVVPPKPWTGITGGGYWANGRRPLALVRTHSKKALMRYED
VYMPEVYKALNIAQNTAWKINKKVLAVANVITKWKHCPVEDIPAIEREELPMKPEDIDMNPE
ALTAWKRAAAAVYRKDKARKSRRISLEFMLEQANKFANHKAIWFPYNMDWRGRVYAVSMFNP
QGNDMTKGLLTLAKGKPIGKEGYYWLKIHGANCAGVDKVPFPERIKFIEENHENIMACAKSP
LENTWWAEQDSPFCFLAFCFEYAGVQHHGLSYNCSLPLAFDGSCSGIQHFSAMLRDEVGGRA
VNLLPSETVQDIYGIVAKKVNEILQADAINGTDNEVVTVTDENTGEISEKVKLGTKALAGQW
LAYGVTRSVTKRSVMTLAYGSKEFGFRQQVLEDTIQPAIDSGKGLMFTQPNQAAGYMAKLIW
ESVSVTVVAAVEAMNWLKSAAKLLAAEVKDKKTGEILRKRCAVHWVTPDGFPVWQEYKKPIQ
TRLNLMFLGQFRLQPTININKDSEIDAHKQESGIAPNFVHSQDGSHLRKTVVWAHEKYGIES
FALIHDSFGTIPADAANLFKAVRETMVDTYESCDVLADFYDQFADQLHESQLDKMPALPAKG
NLNLRDILESDFAFA (SEQ ID NO: 6)
Engineered T7 RNA Polymerase (I396L)
MNTINIAKNDFSDIELAAIPFNTLADHYGERLAREQLALEHESYEMGEARFRKMFERQLKAG
EVADNAAAKPLITILLPKMIARINDWFEEVKAKRGKRPTAFQFLQEIKPEAVAYITIKTTLA
CLISADNITVQAVASAIGRAIEDEARFGRIRDLEAKHFKKNVEEQLNKRVGHVYKKAFMQVV
EADMLSKGLLGGEAWSSWHKEDSIHVGVRCIEMLIESTGMVSLHRQNAGVVGQDSETIELAP
EYAEAIATRAGALAGISPMFQPCVVPPKPWIGITGGGYWANGRRPLALVRTHSKKALMRYED
VYMPEVYKAINIAQNTAWKINKKVLAVANVITKWKHCPVEDIPAIEREELPMKPEDIDMNPE
ALTAWKRAAAAVYRKDKARKSRRLSLEFMLEQANKFANHKAIWFPYNMDWRGRVYAVSMFNP
QGNDMIKGLLTLAKGKPIGKEGYYWLKIHGANCAGVDKVPFPERIKFIEENHENIMACAKSP
LENTWWAEQDSPFCFLAFCFEYAGVQHHGLSYNCSLPLAFDGSCSGIQHFSAMLRDEVGGRA
VNLLPSETVQDIYGIVAKKVNEILQADAINGIDNEVVIVIDENTGEISEKVKLGTKALAGQW
LAYGVIRSVIKRSVMTLAYGSKEFGFRQQVLEDTIQPAIDSGKGLMFTQPNQAAGYMAKLIW
ESVSVIVVAAVEAMNWLKSAAKLLAAEVKDKKTGEILRKRCAVHWVTPDGFPVWQEYKKPIQ
TRLNLMFLGQFRLQPTININKDSEIDAHKQESGIAPNFVHSQDGSHLRKTVVWAHEKYGIES
FALIHDSFGTIPADAANLFKAVRETMVDTYESCDVLADFYDQFADQLHESQLDKMPALPAKG
NLNLRDILESDFAFA (SEQ ID NO: 7)
Engineered T7 RNA Polymerase (F546W)
MNTINIAKNDFSDIELAAIPFNTLADHYGERLAREQLALEHESYEMGEARFRKMFERQLKAG
EVADNAAAKPLITILLPKMIARINDWFEEVKAKRGKRPTAFQFLQEIKPEAVAYITIKTTLA
CLISADNITVQAVASAIGRAIEDEARFGRIRDLEAKHFKKNVEEQLNKRVGHVYKKAFMQVV
EADMLSKGLLGGEAWSSWHKEDSIHVGVRCIEMLIESTGMVSLHRQNAGVVGQDSETIELAP
EYAEAIATRAGALAGISPMFQPCVVPPKPWIGITGGGYWANGRRPLALVRTHSKKALMRYED
VYMPEVYKAINIAQNTAWKINKKVLAVANVITKWKHCPVEDIPAIEREELPMKPEDIDMNPE
ALTAWKRAAAAVYRKDKARKSRRISLEFMLEQANKFANHKAIWFPYNMDWRGRVYAVSMFNP
QGNDMIKGLLTLAKGKPIGKEGYYWLKIHGANCAGVDKVPFPERIKFIEENHENIMACAKSP
LENTWWAEQDSPFCFLAFCFEYAGVQHHGLSYNCSLPLAFDGSCSGIQHWSAMLRDEVGGRA
VNLLPSETVQDIYGIVAKKVNEILQADAINGIDNEVVIVIDENTGEISEKVKLGTKALAGQW
LAYGVIRSVIKRSVMTLAYGSKEFGFRQQVLEDTIQPAIDSGKGLMFTQPNQAAGYMAKLIW
ESVSVIVVAAVEAMNWLKSAAKLLAAEVKDKKTGEILRKRCAVHWVTPDGFPVWQEYKKPIQ
TRLNLMFLGQFRLQPTININKDSEIDAHKQESGIAPNFVHSQDGSHLRKTVVWAHEKYGIES
FALIHDSFGTIPADAANLFKAVRETMVDTYESCDVLADFYDQFADQLHESQLDKMPALPAKG
NLNLRDILESDFAFA (SEQ ID NO: 8)
Engineered T7 RNA Polymerase (S684A)
MNTINIAKNDFSDIELAAIPFNTLADHYGERLAREQLALEHESYEMGEARFRKMFERQLKAG
EVADNAAAKPLITILLPKMIARINDWFEEVKAKRGKRPTAFQFLQEIKPEAVAYITIKTTLA
CLISADNITVQAVASAIGRAIEDEARFGRIRDLEAKHFKKNVEEQLNKRVGHVYKKAFMQVV
EADMLSKGLLGGEAWSSWHKEDSIHVGVRCIEMLIESTGMVSLHRQNAGVVGQDSETIELAP
EYAEAIATRAGALAGISPMFQPCVVPPKPWIGITGGGYWANGRRPLALVRTHSKKALMRYED
VYMPEVYKAINIAQNTAWKINKKVLAVANVITKWKHCPVEDIPAIEREELPMKPEDIDMNPE
ALTAWKRAAAAVYRKDKARKSRRISLEFMLEQANKFANHKAIWFPYNMDWRGRVYAVSMFNP
QGNDMTKGLLTLAKGKPIGKEGYYWLKIHGANCAGVDKVPFPERIKFIEENHENIMACAKSP
LENTWWAEQDSPFCFLAFCFEYAGVQHHGLSYNCSLPLAFDGSCSGIQHFSAMLRDEVGGRA
VNLLPSETVQDIYGIVAKKVNEILQADAINGTDNEVVTVTDENTGEISEKVKLGTKALAGQW
LAYGVTRSVTKRSVMTLAYGSKEFGFRQQVLEDTIQPAIDSGKGLMFTQPNQAAGYMAKLIW
EAVSVTVVAAVEAMNWLKSAAKLLAAEVKDKKTGEILRKRCAVHWVTPDGFPVWQEYKKPIQ
TRLNLMFLGQFRLQPTINTNKDSEIDAHKQESGIAPNFVHSQDGSHLRKTVVWAHEKYGIES
FALIHDSFGTIPADAANLFKAVRETMVDTYESCDVLADFYDQFADQLHESQLDKMPALPAKG
NLNLRDILESDFAFA (SEQ ID NO: 9)
Engineered T7 RNA Polymerase (G788A)
MNTINIAKNDFSDIELAAIPFNTLADHYGERLAREQLALEHESYEMGEARFRKMFERQLKAG
EVADNAAAKPLITTLLPKMIARINDWFEEVKAKRGKRPTAFQFLQEIKPEAVAYITIKTTLA
CLTSADNTTVQAVASAIGRAIEDEARFGRIRDLEAKHFKKNVEEQLNKRVGHVYKKAFMQVV
EADMLSKGLLGGEAWSSWHKEDSIHVGVRCIEMLIESTGMVSLHRQNAGVVGQDSETIELAP
EYAEAIATRAGALAGISPMFQPCVVPPKPWTGITGGGYWANGRRPLALVRTHSKKALMRYED
VYMPEVYKAINIAQNTAWKINKKVLAVANVITKWKHCPVEDIPAIEREELPMKPEDIDMNPE
ALTAWKRAAAAVYRKDKARKSRRISLEFMLEQANKFANHKAIWFPYNMDWRGRVYAVSMFNP
QGNDMTKGLLTLAKGKPIGKEGYYWLKIHGANCAGVDKVPFPERIKFIEENHENIMACAKSP
LENTWWAEQDSPFCFLAFCFEYAGVQHHGLSYNCSLPLAFDGSCSGIQHFSAMLRDEVGGRA
VNLLPSETVQDIYGIVAKKVNEILQADAINGTDNEVVTVTDENTGEISEKVKLGTKALAGQW
LAYGVTRSVTKRSVMTLAYGSKEFGFRQQVLEDTIQPAIDSGKGLMFTQPNQAAGYMAKLIW
ESVSVTVVAAVEAMNWLKSAAKLLAAEVKDKKTGEILRKRCAVHWVTPDGFPVWQEYKKPIQ
TRLNLMFLGQFRLQPTINTNKDSEIDAHKQESGIAPNFVHSQDASHLRKTVVWAHEKYGIES
FALIHDSFGTIPADAANLFKAVRETMVDTYESCDVLADFYDQFADQLHESQLDKMPALPAKG
NLNLRDILESDFAFA (SEQ ID NO: 10)
RNA Polymerase from Bacteriophage T3
(NCBI Reference Sequence: NP_523301.1)
MNIIENIEKNDFSEIELAAIPFNTLADHYGSALAKEQLALEHESYELGERRFLKMLERQAKA
GEIADNAAAKPLLATLLPKLTTRIVEWLEEYASKKGRKPSAYAPLQLLKPEASAFITLKVIL
ASLTSTNMTTIQAAAGMLGKAIEDEARFGRIRDLEAKHFKKHVEEQLNKRHGQVYKKAFMQV
VEADMIGRGLLGGEAWSSWDKETTMHVGIRLIEMLIESTGLVELQRHNAGNAGSDHEALQLA
QEYVDVLAKRAGALAGISPMFQPCVVPPKPWVAITGGGYWANGRRPLALVRTHSKKGLMRYE
DVYMPEVYKAVNLAQNTAWKINKKVLAVVNEIVNWKNCPVADIPSLERQELPPKPDDIDTNE
AALKEWKKAAAGIYRLDKARVSRRISLEFMLEQANKFASKKAIWFPYNMDWRGRVYAVPMFN
PQGNDMTKGLLTLAKGKPIGEEGFYWLKIHGANCAGVDKVPFPERIAFIEKHVDDILACAKD
PINNTWWAEQDSPFCFLAFCFEYAGVTHHGLSYNCSLPLAFDGSCSGIQHFSAMLRDEVGGR
AVNLLPSETVQDIYGIVAQKVNEILKQDAINGTPNEMITVTDKDTGEISEKLKLGTSTLAQQ
WLAYGVTRSVTKRSVMTLAYGSKEFGFRQQVLDDTIQPAIDSGKGLMFTQPNQAAGYMAKLI
WDAVSVTVVAAVEAMNWLKSAAKLLAAEVKDKKTKEILRHRCAVHWTTPDGFPVWQEYRKPL
QKRLDMIFLGQFRLQPTINTLKDSGIDAHKQESGIAPNFVHSQDGSHLRMTVVYAHEKYGIE
SFALIHDSFGTIPADAGKLFKAVRETMVITYENNDVLADFYSQFADQLHETQLDKMPPLPKK
GNLNLQDILKSDFAFA (SEQ ID NO: 11)
RNA polymerase from Bacteriophage 5P6
(UniProt: P06221)
MQDLHAIQLQLEEEMFNGGIRRFEADQQRQIAAGSESDTAWNRRLLSELIAPMAEGIQAY
KEEYEGKKGRAPRALAFLQCVENEVAAYITMKVVMDMLNTDATLQAIAMSVAERIEDQVR
FSKLEGHAAKYFEKVKKSLKASRTKSYRHAHNVAVVAEKSVAEKDADFDRWEAWPKETQL
QIGTTLLEILEGSVFYNGEPVFMRAMRTYGGKTIYYLQTSESVGQWISAFKEHVAQLSPA
YAPCVIPPRPWRTPFNGGFHTEKVASRIRLVKGNREHVRKLTQKQMPKVYKAINALQNTQ
WQINKDVLAVIEEVIRLDLGYGVPSFKPLIDKENKPANPVPVEFQHLRGRELKEMLSPEQ
WQQFINWKGECARLYTAETKRGSKSAAVVRMVGQARKYSAFESIYFVYAMDSRSRVYVQS
STLSPQSNDLGKALLRFTEGRPVNGVEALKWFCINGANLWGWDKKTFDVRVSNVLDEEFQ
DMCRDIAADPLTFTQWAKADAPYEFLAWCFEYAQYLDLVDEGRADEFRTHLPVHQDGSCS
GIQHYSAMLRDEVGAKAVNLKPSDAPQDIYGAVAQVVIKKNALYMDADDATTFTSGSVTL
SGTELRAMASAWDSIGITRSLTKKPVMTLPYGSTRLTCRESVIDYIVDLEEKEAQKAVAE
GRTANKVHPFEDDRQDYLTPGAAYNYMTALIWPSISEVVKAPIVAMKMIRQLARFAAKRN
EGLMYTLPTGFILEQKIMATEMLRVRTCLMGDIKMSLQVETDIVDEAAMMGAAAPNFVHG
HDASHLILTVCELVDKGVTSIAVIHDSFGTHADNTLTLRVALKGQMVAMYIDGNALQKLL
EEHEVRWMVDTGIEVPEQGEFDLNEIMDSEYVFA (SEQ ID NO: 12)
Wild-Type RNA Polymerase from Erwinia phage FE44
(NCBI Reference Sequence: YP_008766719.1)
MTNVINAPKNDFSDIANAIQPYNILADHYGAHLAATQLELEHEAHTEGEKRFLKAMERQIKA
GEFGDNAVAKPLLSSLAPKFIEAWNTWFTEVESKRGKRPVAYNLVQKVAPEAAAFITLKVTL
ACLTKEEYTNLQSVATKIGRSIEDELRFGRIRDEEAKHFKNHVQEALNKRVGIVYKKAFMQA
VEGKMLDAGQLQTKWTTWTPEESIHVGVRMLELLIGSTGLVELHRPFAGNVEKDGEYIQLTE
QYVDLLSKRAGALAAIAPMYQPCVVPPKPWTSPVGGGYWAAGRKPLSLVRTGSKKGLERYND
VYMPEVYKAVNIAQNTPWKINKKVLAVVNEIVNWKHCPVDDVPALERGELPVKPEDIDTNEV
ALKAWKKAASAIYRKEKARVSRRMSMEFMLGQANKFAQFKAIWFPMNMDWRGRVYAVPMFNP
QGNDMTKGLLTLAKGKPIGVDGYYWLKIHGANTAGVDKVDFAERIKFIEDNHENIMSVAADP
IANTWWAEQDSPFCFLAFCFEYAGVQHHGMNYNCSLPLAFDGSCSGIQHFSAMLRDEIGGRA
VNLLPSKEVQDIYRIVAERVNEILKQDVINGTDNEVETVTNKDTGEITEKLKLGTKELAGQW
LAYGVTRKVTKRSVMTLAYGSKEYGFRDQVLEDTIQPAIDDGKGLMFTQPNQAAGYMAKLIW
NAVTVTVVAAVEAMNWLKSAAKLLAAEVKDKKTKEVLRKRCAVHWVTPDGFPVWQEYRKPVQ
TRLNLMFLGQIRLQPTVNTNKDSGIDARKQESGIAPNFVHSMDGSHLRMTVVRSNEVYGVES
FALIHDSFGTIPADAGNLFKAVRETMVNTYEENDVLADFYDQFADQLHESQLDKMPEMPAKG
SLDLQEILKSDFAFA (SEQ ID NO: 13)
RNA polymerase from Kluyvera bacteriophage Kvp1
(GenBank: ACJ14548.1)
MNVINAPKNDFSDIANAIQPYNILADHYGAQLAATQLELEHEAHTEGEKRFLKAMERQIKAG
EFGDNAVAKPLLSSLAPKFIEAWNTWFTEVEAKRGKRPVAYNLVQKVAPEAAAFITLKVTLA
CLTKEEFTNLQSVATKIGRSIEDELRFGRIRDEEAKHFKNHVQEALNKRVGIVYKKAFMQAV
EGKMLDAGQLQTKWTTWTPEESIHVGVRMLELLIGSTGLVELHRPFAGNVEKDGEYIQLTEQ
YVDLLSKRAGALAAIAPMYQPCVVPPKPWTSPVGGGYWAAGRKPLSLVRTGSKKGLERYNDV
YMPEVYKAVNIAQNTPWKINKKVLAVVNEIVNWKHCPVEDVPALERGELPVKPEDIDTNEAA
LKAWKKAASAIYRKEKARVSRRMSMEFMLGQANKFAQFKAIWFPMNMDWRGRVYAVPMFNPQ
GNDMTKGLLTLAKGKPIGVDGYYWLKIHGANTAGVDKVDFAERIKFIDDNHENIMSVAADPI
ANTWWAEQDSPFCFLAFCFEYAGVQHHGMNYNCSLPLAFDGSCSGIQHFSAMLRDEVGGRAV
NLLPSKEVQDIYRIVAERVNEMLREAVINGTDNEVETVTNKDTGEITEKLKLGTKELAGQWL
AYGVTRKVTKRSVMTLAYGSKEYGFRDQVLEDTIQPAIDDGKGLMFTQPNQAAGYMAKLIWE
SVTVTVVAAVEAMNWLKSAAKLLAAEVKDKKTKEVLRKRCAVHWVTPDGFPVWQEYKKPVQT
RLNLMFLGQIRLQPTVNTNKDSGIDARKQESGIAPNFVHSMDGSHLRMTVVRSNEVYGVESF
ALIHDSFGTIPADAGNLFKAVRETMVNTYEENDVLADFYEQFADQLHESQLDKMPEMPAKGS
LDLQEILKSDFAFA (SEQ ID NO: 14)
RNA polymerase from Yersinia bacteriophage phiYeO3-12
(UniProt: Q9T145)
MNIIENIEKNDFSEIELAAIPFNTLADHYGSALAREQLALEHESYELGERRFLKMLERQA
KAGEIADNAAAKPLLATLLPKLTTRIVEWLEEYATKKGRKPVAYAPLQSLKPEASAFITL
KVILASLTSTNMTTIQAAAGMLGKAIEDEARFGRIRDLEAKHFKKHVEEQLNKRHGQVYK
KAFMQVVEADMIGRGLLGGEAWSSWDKETTMHVGIRLIEMLIESTGLVELQRHNAGNAGS
DHEALQLAQEYVDVLAKRAGALAGISPMFQPCVVPPKPWVAITGGGYWANGRRPLALVRT
HSKKGLMRYEDVYMPEVYKAVNIAQNTAWKINKKVLAVVNEIVNWKNCPVADIPSLERQE
LPPKPDDIDTNEAALKEWKKAAAGIYRLDKARVSRRISLEFMLEQANKFASKKAIWFPYN
MDWRGRVYAVPMFNPQGNDMTKGLLTLAKGKPIGEEGFYWLKIHGANCAGVDKVPFPERI
AFIEKHVDDILACAKDPINNTWWAEQDSPFCFLAFCFEYAGVAHHGLSYNCSLPLAFDGS
CSGIQHFSAMLRDEVGGRAVNLLPSETVQDIYGIVAQKVNEILKQDAINGTPNEMITVTD
KDTGEISEKLKLGTSTLAQQWLAYGVTRSVTKRSVMTLAYGSKEFGFRQQVLDDTIQPAI
DSGKGLMFTQPNQAAGYMAKLIWDAVSVTVVAAVEAMNWLKSAAKLLAAEVKDKKTKEIL
RHRCAVHWTTPDGFPVWQEYRKPLQKRLDMIFLGQFRLQPTINTLKDSGIDAHKQESGIA
PNFVHSQDGSHLRMTVVYAHENYGIESFALIHDSFGTIPADAGKLFKAVRETMVITYENN
DVLADFYDQFADQLHETQLDKMPPLPKKGNLNLQDILKSDFAFA (SEQ ID NO: 15)

### Equivalents and Scope

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. A T7 RNA polymerase variant comprising at least three amino acid substitutions in the amino acid sequence of SEQ ID NO: 1 or an amino acid sequence having at least 70% identity to SEQ ID NO: 1, wherein the at least three amino acid substitutions are at positions selected from the group consisting: I320, I396, F546, S684, and G788.

2. The T7 RNA polymerase variant of claim 1, wherein the variant comprises a I320L substitution in the amino acid sequence identified by SEQ ID NO:1.

3. The T7 RNA polymerase variant of claim 1, wherein the variant comprises a I396L substitution in the amino acid sequence identified by SEQ ID NO:1.

4. The T7 RNA polymerase variant of claim 1, wherein the variant comprises a F546W substitution in the amino acid sequence identified by SEQ ID NO:1.

5. The T7 RNA polymerase variant of claim 1, wherein the variant comprises a S684A substitution in the amino acid sequence identified by SEQ ID NO:1.

6. The T7 RNA polymerase variant of claim 1, wherein the variant comprises a G788A substitution in the amino acid sequence identified by SEQ ID NO:1.

7. The T7RNA polymerase variant of claim 1, wherein the variant comprises the substitutions I320L, I396L, F546W, S684A and G788A.

8. The T7 RNA polymerase variant of claim 7, wherein the variant comprises the amino acid sequence identified by SEQ ID NO: 2.

9. The T7 RNA polymerase variant of claim 1 comprising the substitution I320L, I396L, and G788A.

10. The T7 RNA polymerase variant of claim 9 comprising the amino acid sequence identified by SEQ ID NO: 3.

11. The T7 RNA polymerase variant of claim 1 comprising the substitutions I396L, S684A, and G788A.

12. The T7 RNA polymerase variant of claim 11 comprising the amino acid sequence identified by SEQ ID NO: 4.

13. The T7 RNA polymerase variant of claim 1 comprising the substitutions I320L, S684A, and G788A.

14. The T7 RNA polymerase variant of claim 13 comprising the amino acid sequence identified by SEQ ID NO: 5.

15. A method for producing ribonucleic acid (RNA), the method comprising:
combining the T7 RNA polymerase variant of any one of claims 1-14 with nucleoside triphosphates and a deoxyribonucleic acid (DNA) template encoding an RNA of interest; and
producing the RNA of interest.

16. The method of claim 15, wherein the step of producing is performed at a temperature of 37° C. or greater.

17. The method of claim 15 or 16, wherein the step of producing comprises using a cell lysate from cells that express the T7 RNA polymerase variant or an enzyme preparation from cells that express the T7 RNA polymerase variant.

18. The method of any one of claims 15-17, wherein the DNA template is a circular DNA template.

19. The method of any one of claims 15-17, wherein the DNA template is a linear DNA template.

20. The method of any one of claims 15-19, wherein an amount of RNA of interest produced is greater than an amount of the RNA of interest produced using a wild-type T7 RNA polymerase at a temperature of 37° C. or greater.

21. The method of any one of claims 17-20 wherein the RNA of interest is dsRNA, ssRNA, siRNA, miRNA, piRNA, mRNA, or shRNA.

22. A kit comprising:
(i) the T7 RNA polymerase variant of any one of claims 1-14; and
(ii) a reaction buffer.

23. The kit of claim 22, wherein the kit further comprises one or more ribonucleoside triphosphates.
